(19) United States
(12) Patent Application Publication
(10) Pub. No.: US 2025/0213199 A1
(43) Pub. Date: Jul. 3, 2025

(54) HEMODYNAMIC MONITOR FOR TRIAGING PATIENTS WITH LOW EJECTION FRACTION

(71) Applicant: Becton, Dickinson and Company, Franklin Lakes, NJ (US)

(72) Inventors: Arghavan Arafati, Mission Viejo, CA (US); Cristhian M. Potes Blandon, Rancho Santa Margarita, CA (US); Feras Al Hatib, Huntington Beach, CA (US); Sai Prasad Buddi, Hyderabad (IN)

(21) Appl. No.: 19/080,437

(22) Filed: Mar. 14, 2025

Related U.S. Application Data
(63) Continuation of application No. PCT/US2023/032950, filed on Sep. 15, 2023.
(60) Provisional application No. 63/375,843, filed on Sep. 15, 2022.

(51) Int. Cl. A61B 5/00; A61B 5/0225; A61B 5/029; G16H 40/63; G16H 50/70

(52) U.S. Cl. CPC A61B 5/7275; A61B 5/02255; A61B 5/029; A61B 5/7267; A61B 5/742; A61B 5/746; G16H 40/63; G16H 50/70

(57) ABSTRACT

A hemodynamic monitor includes a non-invasive blood pressure sensor and an integrated hardware unit with a system processor, a system memory, and a display with a user interface. The system memory includes instructions that are configured to: adjust, by a pressure controller, a pressure within an inflatable blood pressure bladder to maintain a constant volume of an artery of a patient for a period of time; generate an arterial pressure waveform data of the patient based on the adjusted pressure within the inflatable blood pressure bladder over the period of time; extract a plurality of signal measures from the arterial pressure waveform data of the patient; extract input features from the plurality of signal measures that are indicative of an ejection fraction score of the patient; and determine the ejection fraction score of the patient based on the extracted input features.

### CROSS-REFERENCE TO RELATED APPLICATION(S)

This application is a continuation of PCT Application No. PCT/US2023/032950, filed Sep. 15, 2023, and entitled “HEMODYNAMIC MONITOR FOR TRIAGING PATIENTS WITH LOW EJECTION FRACTION,” which claims the benefit of U.S. Provisional Application No. 63/375,843, filed Sep. 15, 2022, and entitled “HEMODYNAMIC MONITOR FOR TRIAGING PATIENTS WITH LOW EJECTION FRACTION OR AORTIC STENOSIS,” the disclosures of which are hereby incorporated by reference in their entireties.

### BACKGROUND

The present disclosure relates generally to ejection fraction, and in particular to measuring ejection fraction in a patient and triaging a patient for treatment.

Ejection fraction is a measurement of the amount of blood pumped out of a chamber of a heart with each contraction. Ejection fraction essentially compares the amount of blood in the chamber of the heart to the amount of blood pumped out of the chamber of the heart. Left ventricular ejection fraction is the ejection fraction of the left heart and indicates how effectively blood is being pumped by the heart into the systemic circulatory system. Traditionally, ejection fraction of a patient is measured through image tests, such an echocardiogram, a multigated acquisition (MUGA) scan, or a computerized tomography (CT) scan. Other tests used to determine ejection fraction include cardiac catheterization and nuclear stress testing. Each of these tests requires a highly-trained specialist to perform the test and interpret the results of the test. Thus, patients must travel to a cardiologist or other cardiovascular specialist to get an initial heart screening. These tests can be expensive and can possibly take days or weeks to inform the patient of their ejection fraction. A solution is needed that will provide greater access to ejection fraction screening for patients with less travel. Preferably, the solution will also reduce the amount of time patients must wait to get results from their ejection fraction screenings so patients can seek further testing and/or treatment with less delay.

### SUMMARY

In one example, a hemodynamic monitor is disclosed for detecting heart failure. The hemodynamic monitor includes a non-invasive blood pressure sensor comprising an inflatable blood pressure bladder, a pressure controller pneumatically connected to the inflatable blood pressure bladder, and an optical transmitter and an optical receiver that are electrically connected to the pressure controller. The hemodynamic monitor also includes an integrated hardware unit with a system processor, a system memory, and a display with a user interface. The system memory includes instructions that, when executed by the system processor, are configured to: adjust, by the pressure controller, a pressure within the inflatable blood pressure bladder to maintain a constant volume of an artery of a patient for a period of time based on a feedback signal generated by the optical transmitter and the optical receiver; generate an arterial pressure waveform data of the patient based on the adjusted pressure within the inflatable blood pressure bladder over the period of time; extract a plurality of signal measures from the arterial pressure waveform data of the patient; extract input features from the plurality of signal measures that are indicative of an ejection fraction score of the patient; determine the ejection fraction score of the patient based on the extracted input features; generate a first sensory alarm signal configured to generate a first sensory alert that indicates that the patient has low ejection fraction when the ejection fraction score exceeds a threshold score, or generate a second sensory alarm signal configured to generate a second sensory alert that indicates that the patient does not have low ejection fraction when the ejection fraction score is below the threshold score; transmit the first sensory alarm signal or the second sensory alarm signal to the user interface; and output the first sensory alert or the second sensory alert through the user interface.

In another example, a hemodynamic monitor is disclosed for detecting heart failure. The hemodynamic monitor includes an arterial blood pressure sensor with a housing, a fluid input port connected via tubing to a fluid source, a catheter-side fluid port connected to a catheter inserted within an arterial system of a patient, a pressure transducer in communication with the fluid source through the fluid port, and an I/O cable in electrical communication with the pressure transducer. The hemodynamic monitor also includes an integrated hardware unit with a system processor, a system memory, a display comprising a user interface, and an analog-to-digital (ADC) converter. The system memory includes instructions that, when executed by the system processor, are configured to: receive an electrical signal from the pressure transducer over a period of time, the electrical signal based on a pressure of the arterial system of the patient transmitted through the fluid source; convert the electrical signal to a digital signal; generate an arterial pressure waveform data of the patient based on the digital signal; extract a plurality of signal measures from the arterial pressure waveform data; extract input features from the plurality of signal measures that are indicative of an ejection fraction score of the patient; determine the ejection fraction score of the patient based on the extracted input features; generate a first sensory alarm signal configured to generate a first sensory alert that indicates that the patient has low ejection fraction when the ejection fraction score exceeds a threshold score, or generate a second sensory alarm signal configured to generate a second sensory alert that indicates that the patient does not have low ejection fraction when the ejection fraction score is below the threshold score; transmit the first sensory alarm signal or the second sensory alarm signal to the user interface; and output the first sensory alert or the second sensory alert through the user interface.

In a further example, a method for triaging a patient for risk of heart failure is disclosed. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data and extracts input features from the plurality of signal measures that are indicative of an ejection fraction of the patient. The hemodynamic monitor further determines the ejection fraction of the patient based on the input features and outputs the ejection fraction of the patient to a display. The hemodynamic monitor alerts and the patient or medical personnel that the ejection fraction is low when the ejection fraction is less than or equal to forty percent.

### BRIEF DESCRIPTION OF THE DRAWINGS

FIG. 1 is a perspective view of an example hemodynamic monitor that analyzes an arterial pressure of a patient and provides an ejection fraction and a heart failure risk score of a patient to medical personnel.

FIG. 2 is a perspective view of an example minimally invasive pressure sensor for sensing hemodynamic data representative of arterial pressure of a patient.

FIG. 3 is a perspective view of an example non-invasive sensor for sensing hemodynamic data representative of arterial pressure of a patient.

FIG. 4 is a block diagram illustrating an example hemodynamic monitoring system that determines an ejection fraction of a patient based on a set of input features derived from signal measures of an arterial pressure waveform of the patient.

FIG. 5 is a schematic diagram of a method for triaging an ejection fraction of the patient.

FIG. 6 is a diagram of a first clinical dataset, a second clinical dataset, and a third clinical dataset used for data mining and machine training of the hemodynamic monitoring system.

FIG. 7 is a flow diagram for extracting a set of input features derived from signal measures of an arterial pressure waveform of a patient for training a machine learning model of a hemodynamic monitoring system.

FIG. 8 is a graph illustrating an example trace of an arterial pressure waveform including example indicia corresponding to signal measures used to extract the input features that determine the ejection fraction of the patient.

### DETAILED DESCRIPTION

As described herein, a hemodynamic monitoring system uses an arterial waveform of a patient to detect an ejection fraction of a patient. The hemodynamic monitoring system uses machine learning to extract sets of input features from the arterial pressure of the patient. The sets of input features are used by the hemodynamic monitoring system to determine the ejection fraction of the patient while visiting an office of a primary care physician, while in an emergency care setting, or any other patient care environment.

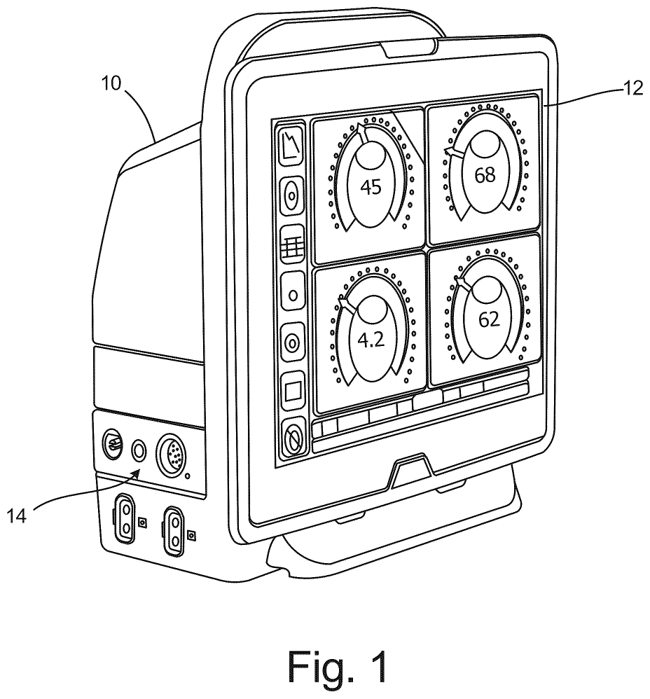

Depending on the ejection fraction measured by the hemodynamic monitoring system, the hemodynamic monitoring system can raise a signal or an alarm to medical workers and/or the patient to alert the medical workers and/or the patient that the ejection fraction of the patient is low and the patient is at high risk for heart failure. The hemodynamic monitoring system is described in detail below with reference to FIGS. 1-8.

FIG. 1 is a perspective view of hemodynamic monitor 10 that can determine an ejection fraction of a patient. As illustrated in FIG. 1, hemodynamic monitor 10 includes display 12 that, in the example of FIG. 1, presents a graphical user interface including control elements (e.g., graphical control elements) that enable user interaction with hemodynamic monitor 10. Hemodynamic monitor 10 can also include a plurality of input and/or output (I/O) connectors configured for wired connection (e.g., electrical and/or communicative connection) with one or more peripheral components, such as one or more hemodynamic sensors, as is further described below. For instance, as illustrated in FIG. 1, hemodynamic monitor 10 can include I/O connectors 14. While the example of FIG. 1 illustrates five separate I/O connectors 14, it should be understood that in other examples, hemodynamic monitor 10 can include fewer than five I/O connectors or greater than five I/O connectors. In yet other examples, hemodynamic monitor 10 may not include I/O connectors 14, but rather may communicate wirelessly with various peripheral devices.

As further described below, hemodynamic monitor 10 includes one or more processors and computer-readable memory that stores ejection fraction software code which is executable to determine an ejection fraction measurement of the patient based on sensed hemodynamic data of the patient. Hemodynamic monitor 10 can receive the sensed hemodynamic data representative of an arterial pressure waveform of the patient, such as via one or more hemodynamic sensors connected to hemodynamic monitor 10 via I/O connectors 14. Hemodynamic monitor 10 executes the ejection fraction software code to obtain, using the sensed hemodynamic data, multiple ejection fraction profiling parameters (e.g., input features), which can include one or more vital sign parameters characterizing vital sign data of the patient, as well as differential and combinatorial parameters derived from the one or more vital sign parameters, as is further described below.

As illustrated in FIG. 1, hemodynamic monitor 10 can present a graphical user interface at display 12. Display 12 can be a liquid crystal display (LCD), a light-emitting diode (LED) display, an organic light-emitting diode (OLED) display, or other display device suitable for providing information to users in graphical form. In some examples, such as the example of FIG. 1, display 12 can be a touch-sensitive and/or presence-sensitive display device configured to receive user input in the form of gestures, such as touch gestures, scroll gestures, zoom gestures, swipe gestures, or other gesture input.

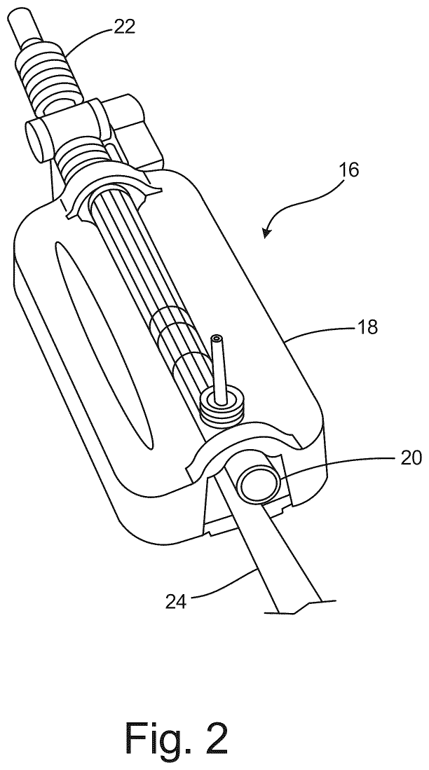

FIG. 2 is a perspective view of hemodynamic sensor 16 that can be attached to the patient for sensing hemodynamic data representative of arterial pressure of the patient. Hemodynamic sensor 16, illustrated in FIG. 2, is one example of a minimally invasive hemodynamic sensor that can be attached to the patient via, e.g., a radial arterial catheter inserted into an arm of the patient. In other examples, hemodynamic sensor 16 can be attached to the patient via a femoral arterial catheter inserted into a leg of the patient.

As illustrated in FIG. 2, hemodynamic sensor 16 includes housing 18, fluid input port 20, catheter-side fluid port 22, and I/O cable 24. Fluid input port 20 is configured to be connected via tubing or other hydraulic connection to a fluid source, such as a saline bag or other fluid input source. Catheter-side fluid port 22 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 24 is configured to connect to hemodynamic monitor 10 via, e.g., one or more of I/O connectors 14 (FIG. 1). Housing 18 of hemodynamic sensor 16 encloses one or more pressure transducers, communication circuitry, processing circuity, and corresponding electronic components to sense fluid pressure corresponding to arterial pressure of the patient that is transmitted to hemodynamic monitor 10 (FIG. 1) via I/O cable 24.

In operation, a column of fluid (e.g., saline solution) is introduced from a fluid source (e.g., a saline bag) through hemodynamic sensor 16 via fluid input port 20 to catheter-side fluid port 22 toward the catheter inserted into the patient. Arterial pressure is communicated through the fluid column to pressure sensors located within housing 16 which sense the pressure of the fluid column. Hemodynamic sensor 16 translates the sensed pressure of the fluid column to an electrical signal via the pressure transducers and outputs the corresponding electrical signal to hemodynamic monitor 10 (FIG. 1) via I/O cable 24. Hemodynamic sensor 16 therefore transmits analog sensor data (or a digital representation of the analog sensor data) to hemodynamic monitor 10 (FIG. 1) that is representative of substantially continuous beat-to-beat monitoring of the arterial pressure of the patient.

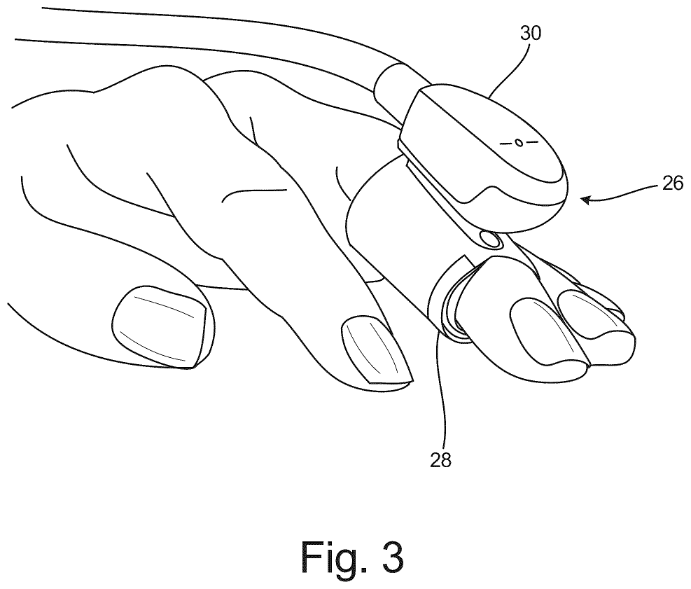

FIG. 3 is a perspective view of hemodynamic sensor 26 for sensing hemodynamic data representative of arterial pressure of the patient. Hemodynamic sensor 26, illustrated in FIG. 3, is one example of a non-invasive hemodynamic sensor that can be attached to the patient via one or more finger cuffs to sense data representative of arterial pressure of the patient. As illustrated in FIG. 3, hemodynamic sensor 26 includes inflatable finger cuff 28 and heart reference sensor 30. Inflatable finger cuff 28 also includes an optical (e.g., infrared) transmitter and an optical receiver that are electrically connected to the pressure controller (not illustrated). The optical transmitter and the optical receiver can measure the changing volume of the arteries under the cuff in the finger. The optical transmitter and the optical receiver can be positioned to transmit and receive light therebetween through the inflatable blood pressure bladder.

In operation, the pressure controller continually adjusts pressure within the finger cuff to maintain a constant volume of the arteries in the finger (i.e., the unloaded volume of the arteries) as measured via the optical transmitter and optical receiver of inflatable finger cuff 28. The pressure applied by the pressure controller to continuously maintain the unloaded volume is representative of the blood pressure in the finger and is communicated by the pressure controller to hemodynamic monitor 10 shown in FIG. 1. Heart reference sensor 30 measures the hydrostatic height difference between the level at which the finger is kept and the reference level for the pressure measurement, which typically is heart level. Accordingly, hemodynamic sensor 26 transmits sensor data that is representative of substantially continuous beat-to-beat monitoring of the arterial pressure waveform of the patient.

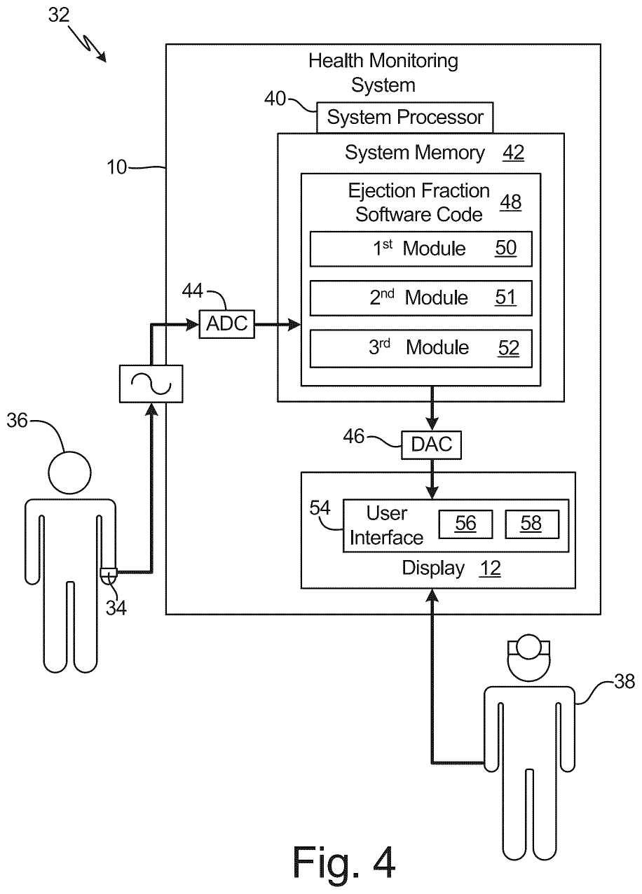

FIG. 4 is a block diagram of hemodynamic monitoring system 32 that determines an ejection fraction measurement of patient 36 based on a set of ejection fraction profiling parameters (also referred to as input features) derived from the arterial pressure of patient 36. Hemodynamic monitoring system 32 monitors the arterial pressure of patient 36 and provides an ejection fraction measurement to medical worker 38. If the ejection fraction measurement of patient 36 is low or borderline, medical worker 38 can respond to the ejection fraction measurement by recommending treatment to patient 36 for heart failure or cardiomyopathy.

As illustrated in FIG. 4, hemodynamic monitoring system 32 includes hemodynamic monitor 10 and hemodynamic sensor 34. Hemodynamic monitoring system 32 can be implemented within an office of a primary care physician during a regular physical or check-up, while in a patient care environment, such as an ICU, an OR, or any other patient care environment. Hemodynamic monitoring system 32 can even be used and operated by patient 36 at home. As illustrated in FIG. 4, the patient care environment can include patient 36 and healthcare worker 38 trained to utilize hemodynamic monitoring system 32.

Hemodynamic monitor 10, as described above with respect to FIG. 1, can be, e.g., an integrated hardware unit including system processor 40, system memory 42, display 12, analog-to-digital (ADC) converter 44, and digital-to-analog (DAC) converter 46. In other examples, any one or more components and/or described functionality of hemodynamic monitor 10 can be distributed among multiple hardware units. For instance, in some examples, display 12 can be a separate display device that is remote from and operatively coupled with hemodynamic monitor 10. In general, though illustrated and described in the example of FIG. 4 as an integrated hardware unit, it should be understood that hemodynamic monitor 10 can include any combination of devices and components that are electrically, communicatively, or otherwise operatively connected to perform the functionality attributed herein to hemodynamic monitor 10.

As illustrated in FIG. 4, system memory 42 stores ejection fraction software code 48. Ejection fraction software code 48 includes first module 50 for extracting and calculating waveform features from the arterial pressure of patient 36, second module 51 for extracting input features from the waveform features, and third module 52 for determining the ejection fraction measurement of patient 36 based on the input features. Display 12 provides user interface 54, which includes control elements 56 that enable user interaction with hemodynamic monitor 10 and/or other components of hemodynamic monitoring system 32. User interface 54, as illustrated in FIG. 4, also provides sensory alarm 58 to provide warning to medical personnel if the ejection fraction of patient 36 is low or borderline. Sensory alarm 58 can be implemented as one or more of a visual alarm, an audible alarm, a haptic alarm, or other type of sensory alarm. For instance, sensory alarm 58 can be invoked as any combination of flashing and/or colored graphics shown by user interface 54 on display 12, display of the ejection fraction measurement via user interface 54 on display 12 along with a heart failure risk score, a warning sound such as a siren or repeated tone, and a haptic alarm configured to cause hemodynamic monitor 10 to vibrate or otherwise deliver a physical impulse perceptible to medical worker 38 or other user.

Hemodynamic sensor 34 can be attached to patient 36 to sense hemodynamic data representative of the arterial pressure waveform of patient 36. Hemodynamic sensor 34 is operatively connected to hemodynamic monitor 10 (e.g., electrically and/or communicatively connected via wired or wireless connection, or both) to provide the sensed hemodynamic data to hemodynamic monitor 10. In some examples, hemodynamic sensor 34 provides the hemodynamic data representative of the arterial pressure waveform of patient 36 to hemodynamic monitor 10 as an analog signal, which is converted by ADC 44 to digital hemodynamic data representative of the arterial pressure waveform. In other examples, hemodynamic sensor 34 can provide the sensed hemodynamic data to hemodynamic monitor 10 in digital form, in which case hemodynamic monitor 10 may not include or utilize ADC 44. In yet other examples, hemodynamic sensor 34 can provide the hemodynamic data representative of the arterial pressure waveform of patient 36 to hemodynamic monitor 10 as an analog signal, which is analyzed in its analog form by hemodynamic monitor 10.

Hemodynamic sensor 34 can be a non-invasive or minimally invasive sensor attached to patient 36. For instance, hemodynamic sensor 34 can take the form of minimally invasive hemodynamic sensor 16 (FIG. 2), non-invasive hemodynamic sensor 26 (FIG. 3), or other minimally invasive or non-invasive hemodynamic sensor. In some examples, hemodynamic sensor 34 can be attached non-invasively at an extremity of patient 36, such as a wrist, an arm, a finger, an ankle, a toe, or other extremity of patient 36. As such, hemodynamic sensor 34 can take the form of a small, lightweight, and comfortable hemodynamic sensor suitable for extended wear by patient 36 to provide substantially continuous beat-to-beat monitoring of the arterial pressure of patient 36 over an extended period of time, such as minutes or possibly hours. While hemodynamic sensor 34 can monitor the arterial pressure of patient 36 over an extended period of time, hemodynamic sensor 34 will only need to monitor the arterial pressure of patient 36 for few minutes (such as 5 minutes) to provide enough data to hemodynamic monitor 10 to determine ejection fraction measurement of patient 36.

In certain examples, hemodynamic sensor 34 can be configured to sense an arterial pressure of patient 36 in a minimally invasive manner. For instance, hemodynamic sensor 34 can be attached to patient 36 via a radial arterial catheter inserted into an arm of patient 36. In other examples, hemodynamic sensor 34 can be attached to patient 36 via a femoral arterial catheter inserted into a leg of patient 36. Such minimally invasive techniques can similarly enable hemodynamic sensor 34 to provide substantially continuous beat-to-beat monitoring of the arterial pressure of patient 36 over an extended period of time, such as minutes or hours. While hemodynamic sensor 34 can monitor the arterial pressure of patient 36 over an extended period of time, hemodynamic sensor 34 will only need to monitor the arterial pressure of patient 36 for few minutes (such as 5 minutes) to provide enough data to hemodynamic monitor 10 to determine ejection fraction measurement of patient 36.

System processor 40 is a hardware processor configured to execute ejection fraction software code 48, which implements first module 50, second module 51, and third module 52 to generate the ejection fraction measurement for patient 36. Examples of system processor 40 can include any one or more of a microprocessor, a controller, a digital signal processor (DSP), an application specific integrated circuit (ASIC), a field-programmable gate array (FPGA), or other equivalent discrete or integrated logic circuitry.

System memory 42 can be configured to store information within hemodynamic monitor 10 during operation. System memory 42, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 42 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Display 12 can be a liquid crystal display (LCD), a light-emitting diode (LED) display, an organic light-emitting diode (OLED) display, or other display device suitable for providing information to users in graphical form. User interface 54 can include graphical and/or physical control elements that enable user input to interact with hemodynamic monitor 10 and/or other components of hemodynamic monitoring system 32. In some examples, user interface 54 can take the form of a graphical user interface (GUI) that presents graphical control elements presented at, e.g., a touch-sensitive and/or presence sensitive display screen of display 12. In such examples, user input can be received in the form of gesture input, such as touch gestures, scroll gestures, zoom gestures, or other gesture input. In certain examples, user interface 54 can take the form of and/or include physical control elements, such as a physical buttons, keys, knobs, or other physical control elements configured to receive user input to interact with components of hemodynamic monitoring system 32.

In operation, hemodynamic sensor 34 senses hemodynamic data representative of an arterial pressure waveform of patient 36. Hemodynamic sensor 34 provides the hemodynamic data (e.g., as analog sensor data), to hemodynamic monitor 10. ADC 44 converts the analog hemodynamic data to digital hemodynamic data representative of the arterial pressure waveform of the patient.

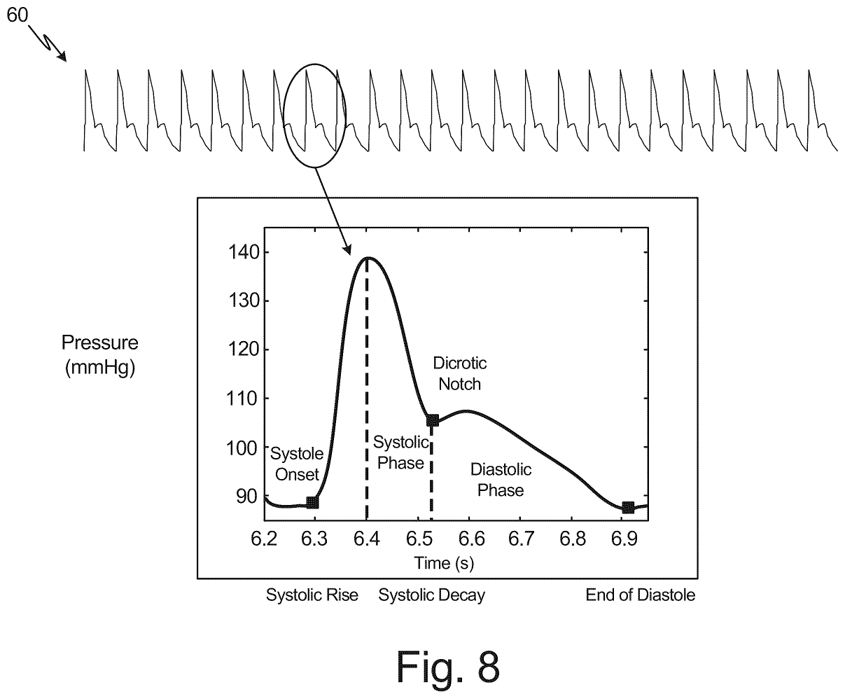

System processor 40 executes ejection fraction software code 48 to determine, using the received hemodynamic data, the ejection fraction measurement for patient 36. For instance, system processor 40 can execute first module 50 to perform waveform analysis of the hemodynamic data to determine a plurality of signal measures. The plurality of signal measures includes waveform features and hemodynamic effects that characterize individual cardiac cycles of the arterial pressure waveform of the patient. The plurality of signal measures is discussed in greater detail below in the discussion of FIG. 8. System processor 40 executes second module 51 to extract input features from the plurality of signal measures that are determinative of the ejection fraction measurement of patient 36. System processor 40 executes third module 52 to determine, based on the input features, the ejection fraction measurement of patient 36. Third module 52 can also convert the ejection fraction measurement of patient 36 into a heart failure score representing a probability of heart failure for patient 36.

If the ejection fraction measurement of patient 36 is equal to or less than forty percent, system processor 40 invokes sensory alarm 58 of user interface 54 to send a first sensory signal to alert medical worker 38 that patient 36 has a low ejection fraction measurement and patient 36 is at high risk of heart failure. Medical worker 38 can respond to the low ejection measurement of patient 36 by recommending patient 36 undergo further tests and examinations to verify the heart health of patient 36. In this manner, hemodynamic monitor 10 functions as a screening tool that can be used in the office of a primary care physician for detecting and discovering heart failure in patient 36 during routine physical examinations. Similarly, hemodynamic monitor 10 can be used at home by patient 36 for self-screening to self-determine whether patient 36 needs to see a doctor or specialist.

If system processor 40 and third module 52 determine that the ejection fraction measurement of patient 36 is within forty-one percent and forty-nine percent, system processor 40 invokes sensory alarm 58 of user interface 54 to send a second sensory signal to alert medical worker 38 that patient 36 has a borderline ejection fraction measurement and patient 36 may be at risk of heart failure in the near future. Medical worker 38 can respond to the borderline ejection measurement of patient 36 by recommending patient 36 undergo further tests and examinations to verify the heart health of patient 36. In this manner, hemodynamic monitor 10 functions as a screening tool that can be used in the office of a primary care physician for detecting a future potential of heart failure, or early onset of heart failure, in patient 36 during routine physical examinations. Similarly, hemodynamic monitor 10 can be used at home by patient 36 for self-screening to self-determine whether patient 36 needs to see a doctor or specialist.

If system processor 40 and third module 52 determine that the ejection fraction measurement of patient 36 is above fifty percent, system processor 40 invokes sensory alarm 58 of user interface 54 to send a third sensory signal to alert medical worker 38 that patient 36 has a normal ejection fraction measurement and that patient 36 has a low risk of heart failure in the near future. Additional screening or examination of patient 36 for ejection fraction is unlikely when hemodynamic monitor 10 determines patient 36 has a normal ejection fraction measurement. As a healthy heart pumps out no more than half to two-thirds the volume of blood in a chamber in one heartbeat, the ejection fraction measurement of patient 36 should not exceed seventy percent.

In some embodiments, system processor 40 can determine multiple subsets of the input features, with each subset of the input features being related to a different level or range of ejection fraction measurement. For example, system processor 40 can execute first module 50 to perform waveform analysis of the hemodynamic data to determine a plurality of signal measures. System processor 40 executes second module 51 to extract a first subset, a second subset, and a third subset of the input features from the plurality of signal measures of patient 36. The first subset of the input features are those input features that third module 52 will use to determine whether patient 36 has a normal ejection fraction measurement. The second subset of the input features are those input features that third module 52 will use to determine whether patient 36 has a low ejection fraction measurement. The third subset of the input features are those input features that third module 52 will use to determine whether patient 36 has a borderline ejection fraction measurement. System processor 40 can execute first module 50 to extract a single batch of the plurality of signal measures for a given unit of time, and that single batch of signal measures can be used by second module 51 to extract all of the first subset, the second subset, and the third subset of the input features for that unit of time. Second module 51 can extract all of the first subset, the second subset, and the third subset of the input features concurrently from the plurality of signal measures. System processor 40 can execute third module 52 to concurrently calculate probabilities of a normal ejection fraction score, a low ejection fraction score, and a borderline ejection fraction score for patient 36.

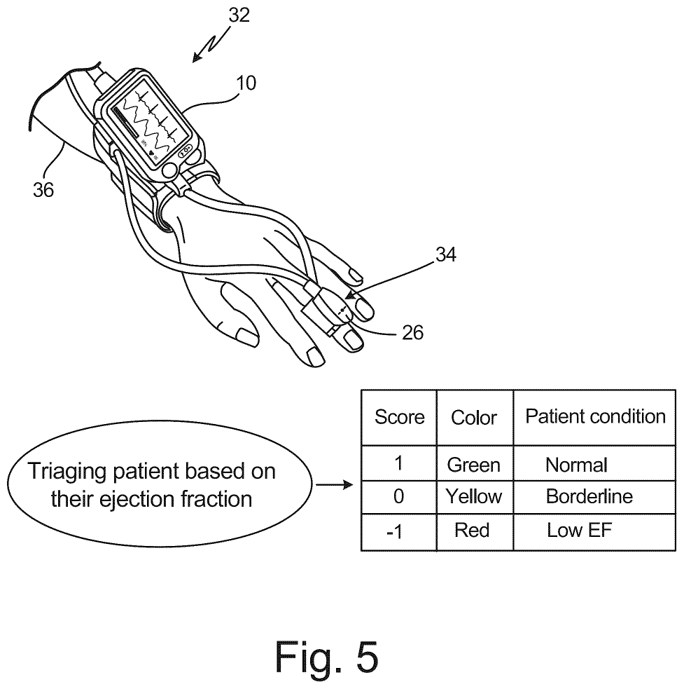

Ejection fraction software code 48 of hemodynamic monitor 10 can utilize, in some examples, a multi-classification-type machine learning model with three labels: normal ejection fraction verses low ejection fraction versus borderline ejection fraction. For example, processor 40 can output to display 12 (and/or a display of a mobile device of patient 36) the normal ejection fraction score of patient 36 with both the low ejection fraction score and the borderline ejection fraction score of patient 36, so that all subset of probabilities are compared together: the probability patient 36 has a normal ejection fraction measurement, the probability that patient 36 has a low ejection fraction measurement, and the probability that patient 36 has a borderline ejection fraction measurement. With the normal ejection fraction score, the low ejection fraction score, and the borderline ejection fraction score of patient 36 together on display 12 of hemodynamic monitor 10, medical worker 38 can better understand and cross-check whether patient 36 has a normal ejection fraction measurement versus a low ejection fraction measurement or borderline ejection fraction measurement. As discussed below with reference to FIG. 5, hemodynamic monitor 10 is a fast and efficient tool for screening and triaging patients 36 before referring patients 36 for more lengthy and costly examination.

FIG. 5 shows a perspective view of hemodynamic monitoring system 32 and a schematic diagram of a method for triaging patient 36 based on the ejection fraction measurement of patient 36. As shown in FIG. 5, hemodynamic monitoring system 32 includes hemodynamic monitor 10 and hemodynamic sensor 34. In the embodiment of FIG. 5, hemodynamic sensor 34 is non-invasive hemodynamic sensor 26 (described in detail above with reference to FIG. 3) that can be attached to patient 36 via one or more finger cuffs to sense data representative of arterial pressure of patient 36. In the embodiment of FIG. 5, hemodynamic monitor 10 is a compact wearable unit that can be strapped to an arm of patient 36 and connected to hemodynamic sensor 34 to receive the sensed data representative of arterial pressure of patient 36. The embodiment of hemodynamic monitoring system 32 of FIG. 5 can operate and function as described above with reference to FIG. 4 to determine the ejection fraction measurement of patient 36. During a routine physical at an office of a primary care physician, patient 36 can be quickly tested and triaged for heart failure risk by connecting hemodynamic monitoring system 32 to a hand and arm of patient 36 and feeding the sensed hemodynamic data of patient 36 into hemodynamic monitor 10. After a few minutes (such as five minutes) of feeding the sensed hemodynamic data of patient 36 into hemodynamic monitor 10, hemodynamic monitor 10 will output to display 12 the ejection fraction measurement of patient 36. In some embodiments, hemodynamic monitor 10 can also output the ejection fraction measurement of patient 36 to a display on a mobile device of patient 36.

Hemodynamic monitor 10 can color-code and/or score the ejection fraction measurement of patient 36 in display 12 depending on whether the ejection fraction measurement is normal, borderline, or low. As noted above with reference to FIG. 4, a normal ejection fraction measurement is above fifty percent, a borderline ejection fraction measurement is within forty-one percent and forty-nine percent, and a low ejection fraction measurement is forty percent or lower. If hemodynamic monitor 10 determines that patient 36 has a normal ejection fraction measurement, hemodynamic monitor 10 can output a green-colored score with a positive numerical value to display 12. If hemodynamic monitor 10 determines that patient 36 has a borderline ejection fraction measurement, hemodynamic monitor can output a yellow-colored score with a neutral or zero numerical value to display 12. If hemodynamic monitor 10 determines that patient 36 has a low ejection fraction measurement, hemodynamic monitor 10 can output a red-colored score with a negative numerical value to display 12.

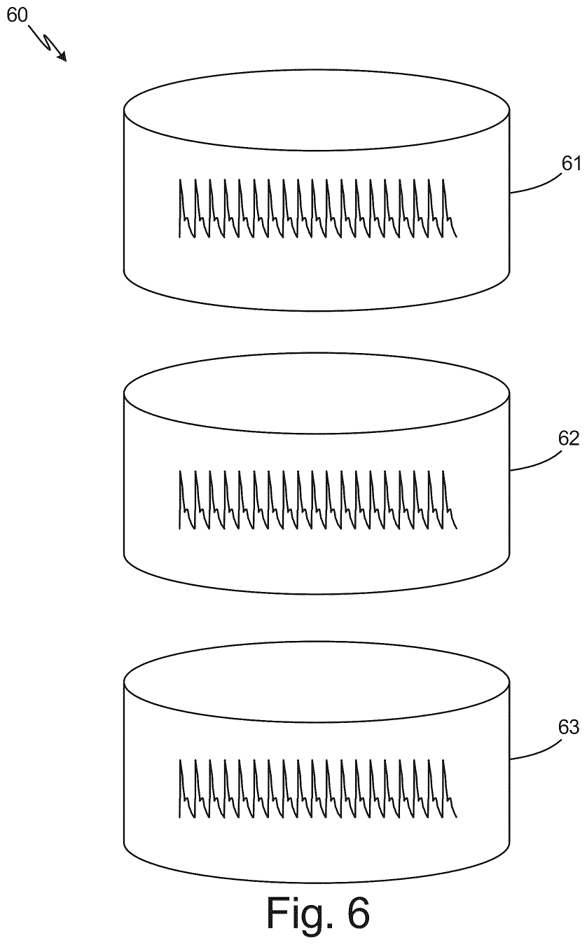

Medical worker 38 (shown in FIG. 4) in this scenario can be a primary care physician or nurse that is performing the routine physical examination of patient 36. Once hemodynamic monitor 10 outputs the ejection fraction measurement of patient 36 to display 12 after monitoring and processing the sensed hemodynamic data of patient 36 for several minutes, medical worker 38 can triage patient 36 based on the ejection fraction measurement of patient 36. If the ejection fraction measurement of patient 36 is indicated as being low on display 12, medical worker 38 can inform patient 36 that patient 36 may be experiencing heart failure and that patient 36 should seek further testing, examination, and treatment immediately from a cardiovascular specialist. Medical worker 38 can then refer patient 36 to a cardiovascular specialist to undergo more intensive examination, such as an echocardiogram, a multigated acquisition (MUGA) scan, a computerized tomography (CT) scan, cardiac catheterization, and/or nuclear stress testing. If the ejection fraction measurement of patient 36 in display 12 is borderline, medical worker 38 can inform patient 36 that patient 36 may be at risk of heart failure and that patient 36 should seek further testing, examination, and treatment in a reasonable amount of time from a cardiovascular specialist. If the ejection fraction measurement of patient 36 in display 12 is normal, medical worker 38 can inform patient 36 that patient 36 is at low risk of heart failure and that no additional testing is needed. As discussed below with reference to FIGS. 6-8, a machine learning model of hemodynamic monitor 10 can be trained using clinical data sets to recognize the input features in the arterial pressure waveform of patient 36 and use those input features to determine the ejection fraction measurement of patient 36.

FIG. 6 is a diagram of clinical data 60 used for data mining and machine training of hemodynamic monitor 10 of hemodynamic monitoring system 32. Clinical data 60 includes first clinical dataset 61, second clinical dataset 62, and third clinical dataset 63.

First clinical dataset 61 contains a collection of arterial pressure waveforms recorded from a first group of individuals who each have a confirmed normal ejection fraction measurement above fifty percent. First clinical dataset 61 can be collected from the first group of individuals by invasive hemodynamic sensors, such as hemodynamic sensor 16 shown in FIG. 2, or collected by non-invasive hemodynamic sensors, such as hemodynamic sensor 26 shown in FIG. 3. When each individual in the first clinical dataset 61 is connected to a hemodynamic sensor, the hemodynamic sensor records an arterial pressure waveform of that individual and the arterial pressure waveform of that individual is tagged with a first label so that arterial pressure waveform can eventually be added to first clinical dataset 61. Adding the first label to the arterial pressure waveforms of the individuals in the first group also allows first clinical dataset 61 to be collected and stored in a common location with second clinical dataset 62 and third clinical dataset 63 without the arterial pressure waveforms of first clinical dataset 61 being lost or confused amongst the arterial pressure waveforms of second clinical dataset 62 and third clinical dataset 63.

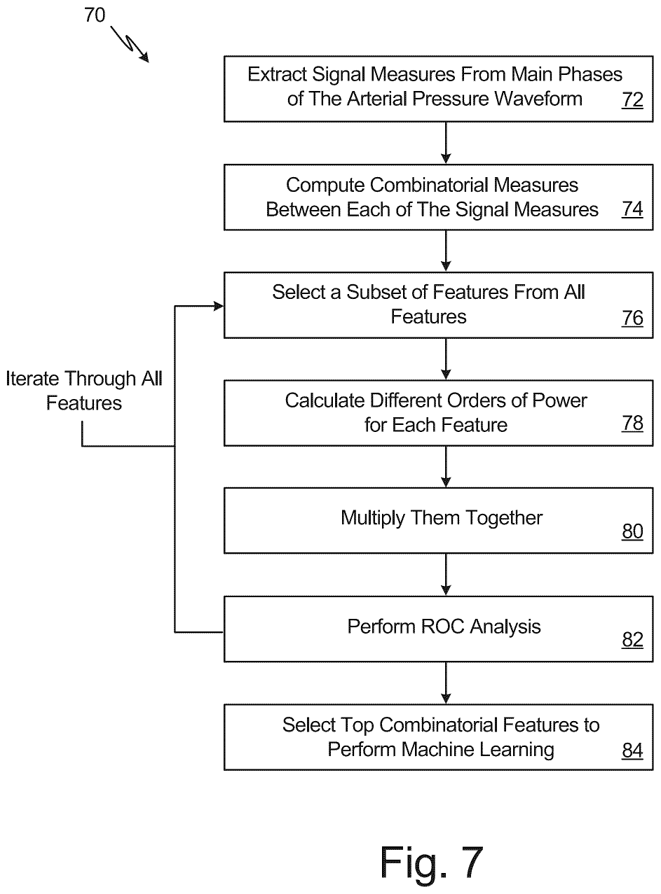

After the arterial pressure waveforms of first clinical dataset 61 have been collected and labeled with first label, the arterial pressure waveforms of first clinical dataset 61 are ready for use for data mining and machine training of hemodynamic monitor 10. The arterial pressure waveforms of first clinical dataset 61 are data mined and used to machine train hemodynamic monitor 10 to determine the first subset of the input features. As discussed above with reference to FIG. 4, the first subset of the input features are those input features that third module 52 will use to determine whether patient 36 has a normal ejection fraction measurement. As will be discussed further below with reference to FIGS. 7-8, waveform analysis is performed on first clinical dataset 61 to calculate a plurality of signal measures which are then used to compute the first subset of the input features that best detect and measure normal ejection fraction from an arterial pressure waveform.

Second clinical dataset 62 contains a collection of arterial pressure waveforms recorded from a second group of individuals who each have a confirmed low ejection fraction measurement that is less than or equal to forty percent. Second clinical dataset 62 can be collected from the second group of individuals by invasive hemodynamic sensors, such as hemodynamic sensor 16 shown in FIG. 2, or collected by non-invasive hemodynamic sensors, such as hemodynamic sensor 26 shown in FIG. 3. When each individual in the second clinical dataset 62 is connected to a hemodynamic sensor, the hemodynamic sensor records an arterial pressure waveform of that individual and the arterial pressure waveform of that individual is tagged with a second label so that arterial pressure waveform can eventually be added to second clinical dataset 62. Adding the second label to the arterial pressure waveforms of the individuals in the second group also allows second clinical dataset 62 to be collected and stored in a common location with first clinical dataset 61 and third clinical dataset 63 without the arterial pressure waveforms of second clinical dataset 62 being lost or confused amongst the arterial pressure waveforms of first clinical dataset 61 and third clinical dataset 63.

After the arterial pressure waveforms of second clinical dataset 62 have been collected and labeled with second label, the arterial pressure waveforms of second clinical dataset 62 are ready for use for data mining and machine training of hemodynamic monitor 10. The arterial pressure waveforms of second clinical dataset 62 are data mined and used to machine train hemodynamic monitor 10 to determine the second subset of the input features. As discussed above with reference to FIG. 4, the second subset of the input features are those input features that third module 52 will use to determine whether patient 36 has a low ejection fraction measurement. As will be discussed further below with reference to FIGS. 7-8, waveform analysis is performed on second clinical dataset 62 to calculate a plurality of signal measures which are then used to compute the second subset of the input features that best detect and measure low ejection fraction from an arterial pressure waveform.

Third clinical dataset 63 contains a collection of arterial pressure waveforms recorded from a third group of individuals who each have a confirmed borderline ejection fraction measurement within forty-one percent and forty-nine percent. Third clinical dataset 63 can be collected from the third group of individuals by invasive hemodynamic sensors, such as hemodynamic sensor 16 shown in FIG. 2, or collected by non-invasive hemodynamic sensors, such as hemodynamic sensor 26 shown in FIG. 3. When each individual in the third clinical dataset 63 is connected to a hemodynamic sensor, the hemodynamic sensor records an arterial pressure waveform of that individual and the arterial pressure waveform of that individual is tagged with a third label so that arterial pressure waveform can eventually be added to third clinical dataset 63. Adding the third label to the arterial pressure waveforms of the individuals in the third group also allows third clinical dataset 63 to be collected and stored in a common location with first clinical dataset 61 and second clinical dataset 62 without the arterial pressure waveforms of third clinical dataset 63 being lost or confused amongst the arterial pressure waveforms of first clinical dataset 61 and second clinical dataset 62.

After the arterial pressure waveforms of third clinical dataset 63 have been collected and labeled with third label, the arterial pressure waveforms of third clinical dataset 62 are ready for use for data mining and machine training of hemodynamic monitor 10. The arterial pressure waveforms of Third clinical dataset 63 are data mined and used to machine train hemodynamic monitor 10 to determine the third subset of the input features. As discussed above with reference to FIG. 4, the third subset of the input features are those input features that third module 52 will use to determine whether patient 36 has a borderline ejection fraction measurement. As will be discussed further below with reference to FIGS. 7-8, waveform analysis is performed on third clinical dataset 62 to calculate a plurality of signal measures which are then used to compute the third subset of the input features that best detect and measure borderline ejection fraction from an arterial pressure waveform.

FIG. 7 is a flow diagram of method 70 for data mining clinical data 60 from FIG. 6 for machine training the machine learning model of hemodynamic monitor 10. Method 70 in FIG. 7 will be discussed while also referencing FIG. 8. Method 70 is applied to each of first clinical dataset 61, second clinical dataset 62, and third clinical dataset 63 to train hemodynamic monitor to find the input features (including the first subset, the second subset, and the third subset of the input features) previously described with reference to FIGS. 4 and 6. Method 70 will be described as applied to the arterial pressure waveforms of first clinical dataset 61.

To machine train hemodynamic monitor 10 to identify the first subset of the input features described in FIG. 4, the first subset of input features is first determined by applying method 70 to the arterial pressure waveforms of first clinical dataset 61 of clinical data 60. First step 72 of method 70 is to perform waveform analysis of the arterial pressure waveforms collected in first clinical dataset 61 to calculate a plurality of signal measures of first clinical dataset 61. Performing waveform analysis of the arterial pressure waveforms of first clinical dataset 61 can include identifying individual cardiac cycles in each of the arterial pressure waveforms of first clinical dataset 61. FIG. 8 provides an example graph illustrating an example trace of an arterial pressure waveform with an individual cardiac cycle identified and enlarged. Next, performing waveform analysis of the arterial pressure waveforms of first clinical dataset 61 can include identifying a dicrotic notch in each of the individual cardiac cycles of each of the arterial pressure waveforms of first clinical dataset 61, similar to the example shown in FIG. 8. Next, the waveform analysis on the arterial pressure waveforms of first clinical dataset 61 includes identifying a systolic rise phase, a systolic decay phase, and a diastolic phase in each of the individual cardiac cycles of each of the arterial pressure waveforms of first clinical dataset 61, similar to the example shown in FIG. 8.

Signal measures are extracted from each of the systolic rise phase, the systolic decay phase, and the diastolic phase from each of the individual cardiac cycles of each of the arterial pressure waveforms of first clinical dataset 61. The signal measures can correspond to hemodynamic effects from each of the systolic rise phase, the systolic decay phase, and the diastolic phase from each of the individual cardiac cycles. Those hemodynamic effects can include contractility, aortic compliance, stroke volume, vascular tone, afterload, and full cardiac cycle. The signal measures calculated or extracted by the waveform analysis of first step 72 of method 70 includes a mean, a maximum, a minimum, a duration, an area, a standard deviation, derivatives, and/or morphological measures from each of the systolic rise phase, the systolic decay phase, and the diastolic phase from each of the individual cardiac cycles. The signal measures can also include heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles of each of the arterial pressure waveforms of first clinical dataset 61.

After the signal measures are determined for first clinical dataset 61, step 74 of method 70 is performed on the signal measures of first clinical dataset 61. Step 74 of method 70 computes combinatorial measures between the signal measures of first clinical dataset 61. Computing the combinatorial measures between the signal measures of first clinical dataset 61 can include performing steps 76, 78, 80, and 82 shown in FIG. 7 on all the signal measures of first clinical dataset 61. Step 76 is performed by arbitrarily selecting a subset of signal measures (such as subset of signal measures) from the signal measures of first clinical dataset 61. Next, different orders of power are calculated for each of the subset of signal measures in the subset of signal measures to generate powers of the subset of signal measures, as shown in step 78 of FIG. 7. In step 80 of FIG. 7, the powers of the subset of signal measures are then multiplied together to generate the product of the powers of the subset of signal measures. Step 82 includes performing receiver operating characteristic (ROC) analysis of the product to arrive at a combinatorial measure for the subset of signal measures. Steps 76, 78, 80, and 82 are repeated until all of the combinatorial measures have been computed between all of the signal measures of first clinical dataset 61. The final step 84 includes selecting the signal measures with most predictive top combinatorial measures (i.e., combinatorial measures satisfying a threshold prediction criteria) as top signal measures for first clinical dataset 61 and are labeled as the first subset of the input features. With the first subset of the input features determined, hemodynamic monitor 10 is trained or programmed to perform waveform analysis on the arterial pressure waveform of patient 36 (shown in FIG. 4) and extract the first subset of the input features from the arterial pressure waveform of patient 36, and use the first subset of the input features to determine whether patient 36 has a normal ejection fraction measurement.

Similar to how method 70 was applied to the arterial pressure waveforms of first clinical dataset 61 to determine the first subset of the input features, method 70 is applied to second clinical dataset 62 to determine the second subset of the input features. Method 70 is also applied to third clinical dataset 63 to determine the third subset of the input features.

### Discussion of Possible Embodiments

The following are non-exclusive descriptions of possible embodiments of the present invention.

In one example, a method for triaging a patient for risk of heart failure includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. The hemodynamic monitor extracts input features from the plurality of signal measures that are indicative of an ejection fraction of the patient. The hemodynamic monitor determines the ejection fraction of the patient based on the input features and outputs the ejection fraction of the patient to a display. The hemodynamic monitor alerts the patient that the ejection fraction is low when the ejection fraction is less than or equal to forty percent. The hemodynamic monitor alerts the patient that the ejection fraction is borderline when the ejection fraction is within forty-one percent and forty-nine percent. The hemodynamic monitor alerts the patient that the ejection fraction is normal when the ejection fraction is above fifty percent.

The method of the preceding paragraph can optionally include, additionally and/or alternatively, any one or more of the following features, configurations, and/or additional components listed below.

A further embodiment of the foregoing method, further comprising: training the hemodynamic monitor for determining the ejection fraction of the patient, wherein training the hemodynamic monitor comprises: collecting a first clinical dataset containing arterial pressure waveforms from a first group of individuals with a normal ejection fraction measurement above; collecting a second clinical dataset containing arterial pressure waveforms from a second group of individuals with a low ejection fraction measurement that is less than or equal to forty percent; collecting a third clinical dataset containing arterial pressure waveforms from a third group of individuals with a borderline ejection fraction measurement within forty-one percent and forty-nine percent; performing waveform analysis of the arterial pressure waveforms of the first clinical dataset, the second clinical dataset, and the third clinical dataset to calculate a plurality of waveform signal measures; and determining the input features by computing combinatorial measures between the plurality of waveform signal measures and selecting top signal measures from the plurality of waveform signal measures with most predictive combinatorial measures and labeling the top signal measures as the input features.

In another example, a system for triaging a patient for risk of heart failure includes a hemodynamic sensor that produces hemodynamic data representative of an arterial pressure waveform of the patient. The system also includes a user interface with a display to show an ejection fraction measurement of the patient to medical personnel. Ejection fraction software code is stored on a system memory of the system. The system includes a processor that is configured to execute the ejection fraction software code to perform: waveform analysis of the hemodynamic data to determine a plurality of signal measures; extract input features from the plurality of signal measures that are indicative of the ejection fraction measurement of the patient; determine, based on the input features, the ejection fraction measurement of the patient; and output the ejection fraction measurement to the display of the user interface.

The system of the preceding paragraph can optionally include, additionally and/or alternatively, any one or more of the following features, configurations, and/or additional components listed below.

A further embodiment of the foregoing system, wherein the input features of the ejection fraction software code are determined by machine training, and wherein the machine training comprises: collecting a first clinical dataset containing arterial pressure waveforms from a first group of individuals with a normal ejection fraction measurement above fifty percent; collecting a second clinical dataset containing arterial pressure waveforms from a second group of individuals with a low ejection fraction measurement less than or equal to forty percent; collecting a third clinical dataset containing arterial pressure waveforms from a third group of individuals with a borderline ejection fraction measurement within forty-one percent and forty-nine percent; performing waveform analysis of the arterial pressure waveforms of the first clinical dataset, the second clinical dataset, and the third clinical dataset to calculate a plurality of waveform signal measures; and determining the input features by computing combinatorial measures between the plurality of waveform signal measures and selecting top signal measures from the plurality of waveform signal measures with most predictive combinatorial measures and labeling the top signal measures as the input features.

A further embodiment of the foregoing system, wherein performing waveform analysis of the labeled arterial pressure waveforms of the first clinical dataset, the second clinical dataset, and the third clinical dataset to calculate the plurality of waveform signal measures comprises: identifying individual cardiac cycles in each of the arterial pressure waveforms of the first clinical dataset, the second clinical dataset, and the third clinical dataset; identifying a dicrotic notch in each of the individual cardiac cycles; identifying a systolic rise phase, a systolic decay phase, and a diastolic phase in each of the individual cardiac cycles; and extracting the plurality of waveform signal measures from each of the systolic rise phase, the systolic decay phase, and the diastolic phase from each of the individual cardiac cycles.

A further embodiment of the foregoing system, wherein the plurality of waveform signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

A further embodiment of the foregoing system, wherein the plurality of waveform signal measures comprises a mean, a maximum, a minimum, a duration, an area, a standard deviation, derivatives, and/or morphological measures from each of the systolic rise phase, the systolic decay phase, and the diastolic phase from each of the individual cardiac cycles.

A further embodiment of the foregoing system, wherein the plurality of waveform signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

A further embodiment of the foregoing system, wherein computing the combinatorial measures between the plurality of waveform signal measures of the first clinical dataset, the second clinical dataset, and the third clinical dataset comprises: performing step one by arbitrarily selecting subset of signal measures from the plurality of waveform signal measures; performing step two by calculating different orders of power for each of the subset of signal measures to generate powers of the subset of signal measures; performing step three by multiplying the powers of the subset of signal measures together to generate the product of the powers of the subset of signal measures; performing step four by performing receiver operating characteristic (ROC) analysis of the product to arrive at a combinatorial measure for the subset of signal measures; and repeating steps one, two, three, and four until all of the combinatorial measures have been computed between all of the plurality of waveform signal measures.

A further embodiment of the foregoing system, wherein the hemodynamic sensor is a noninvasive hemodynamic sensor that is attachable to an extremity of the patient.

A further embodiment of the foregoing system, wherein the hemodynamic sensor is a minimally invasive arterial catheter based hemodynamic sensor.

A further embodiment of the foregoing system, wherein the hemodynamic sensor produces the hemodynamic data as an analog hemodynamic sensor signal representative of the arterial pressure waveform of the patient.

A further embodiment of the foregoing system, further comprising: an analog-to-digital converter that converts the analog hemodynamic sensor signal to digital hemodynamic data representative of the arterial pressure waveform of the patient.

In another example, a method is disclosed for triaging a patient for risk of heart failure. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. The hemodynamic monitor extracts input features from the plurality of signal measures that are indicative of an ejection fraction of the patient. The hemodynamic monitor determines the ejection fraction of the patient based on the input features and outputs the ejection fraction of the patient to a display and/or mobile device. The hemodynamic monitor alerts the patient or medical personnel that the ejection fraction is low when the ejection fraction is less than or equal to forty percent.

The method of the preceding paragraph can optionally include, additionally and/or alternatively, any one or more of the following features, configurations, and/or additional components listed below.

A further embodiment of the foregoing method, further comprising: alerting the patient or the medical personnel that the ejection fraction is borderline when the ejection fraction is within forty-one percent and forty-nine percent.

A further embodiment of the foregoing method, further comprising: alerting the patient or the medical personnel that the ejection fraction is normal when the ejection fraction is above fifty percent.

A further embodiment of the foregoing method, further comprising: training the hemodynamic monitor for determining the ejection fraction of the patient, wherein training the hemodynamic monitor comprises: collecting a first clinical dataset containing arterial pressure waveforms from a first group of individuals with a normal ejection fraction measurement above fifty percent; labeling each of the arterial pressure waveforms of the first clinical dataset with a first label; performing waveform analysis of the labeled arterial pressure waveforms of the first clinical dataset to calculate a plurality of waveform signal measures of the first clinical dataset; and determining a first subset of the input features by computing combinatorial measures between the plurality of waveform signal measures of the first clinical dataset and selecting top signal measures from the plurality of waveform signal measures of the first clinical dataset with most predictive combinatorial measures and labeling the top signal measures of the first clinical data set as the first subset of the input features.

A further embodiment of the foregoing method, wherein training the hemodynamic monitor for determining the ejection fraction of the patient further comprises: collecting a second clinical dataset containing arterial pressure waveforms from a second group of individuals with a low ejection fraction measurement less than or equal to forty percent; labeling each of the arterial pressure waveforms of the second clinical dataset with a second label; performing waveform analysis of the labeled arterial pressure waveforms of the second clinical dataset to calculate a plurality of waveform signal measures of the second clinical dataset; and determining a second subset of the input features by computing combinatorial measures between the plurality of waveform signal measures of the second clinical dataset and selecting top signal measures from the plurality of waveform signal measures of the second clinical dataset with most predictive combinatorial measures and labeling the top signal measures of the second clinical data set as the second subset of the input features.

A further embodiment of the foregoing method, wherein training the hemodynamic monitor for determining the ejection fraction of the patient further comprises: collecting a third clinical dataset containing arterial pressure waveforms from a third group of individuals with a borderline ejection fraction measurement within forty-one percent and forty-nine percent; labeling each of the arterial pressure waveforms of the third clinical dataset with a third label; performing waveform analysis of the labeled arterial pressure waveforms of the third clinical dataset to calculate a plurality of waveform signal measures of the third clinical dataset; and determining a third subset of the input features by computing combinatorial measures between the plurality of waveform signal measures of the third clinical dataset and selecting top signal measures from the plurality of waveform signal measures of the third clinical dataset with most predictive combinatorial measures and labeling the top signal measures of the third clinical data set as the third subset of the input features.

A further embodiment of the foregoing method, wherein performing waveform analysis of the labeled arterial pressure waveforms of the first clinical dataset to calculate the plurality of waveform signal measures of the first clinical dataset comprises: identifying individual cardiac cycles in each of the arterial pressure waveforms of the first clinical dataset; identifying a dicrotic notch in each of the individual cardiac cycles in each of the arterial pressure waveforms of the first clinical dataset; identifying a systolic rise phase, a systolic decay phase, and a diastolic phase in each of the individual cardiac cycles in each of the arterial pressure waveforms of the first clinical dataset; and extracting the plurality of waveform signal measures of the first clinical dataset from each of the systolic rise phase, the systolic decay phase, and the diastolic phase from each of the individual cardiac cycles in each of the arterial pressure waveforms of the first clinical dataset.

A further embodiment of the foregoing method, wherein the plurality of waveform signal measures of the first clinical dataset correspond to hemodynamic effects from each of the systolic rise phase, the systolic decay phase, and the diastolic phase from each of the individual cardiac cycles in each of the arterial pressure waveforms of the first clinical dataset, and wherein the hemodynamic effects comprise contractility, aortic compliance, stroke volume, vascular tone, afterload, and full cardiac cycle.

A further embodiment of the foregoing method, wherein the plurality of waveform signal measures of the first clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the first clinical dataset.

A further embodiment of the foregoing method, wherein the plurality of waveform signal measures of the first clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the first clinical dataset.

A further embodiment of the foregoing method, wherein computing the combinatorial measures between the plurality of waveform signal measures of the first clinical dataset: performing step one by arbitrarily selecting subset of signal measures from the plurality of waveform signal measures of the first clinical dataset; performing step two by calculating different orders of power for each of the subset of signal measures from the plurality of waveform signal measures of the first clinical dataset to generate powers of the subset of signal measures from the plurality of waveform signal measures of the first clinical dataset; performing step three by multiplying the powers of the subset of signal measures from the plurality of waveform signal measures of the first clinical dataset together to generate a product of the powers of the subset of signal measures from the plurality of waveform signal measures of the first clinical dataset; performing step four by performing receiver operating characteristic (ROC) analysis of the product of the powers of the subset of signal measures from the plurality of waveform signal measures of the first clinical dataset to arrive at a combinatorial measure for the subset of signal measures product of the powers of the subset of signal measures from the plurality of waveform signal measures of the first clinical dataset; and repeating steps one, two, three, and four until all of the combinatorial measures have been computed between all of the plurality of waveform signal measures of the first clinical dataset.

A further embodiment of the foregoing method, wherein performing waveform analysis of the labeled arterial pressure waveforms of the second clinical dataset to calculate the plurality of waveform signal measures of the second clinical dataset comprises: identifying individual cardiac cycles in each of the arterial pressure waveforms of the second clinical dataset; identifying a dicrotic notch in each of the individual cardiac cycles in each of the arterial pressure waveforms of the second clinical dataset; identifying a systolic rise phase, a systolic decay phase, and a diastolic phase in each of the individual cardiac cycles in each of the arterial pressure waveforms of the second clinical dataset; and extracting the plurality of waveform signal measures of the second clinical dataset from each of the systolic rise phase, the systolic decay phase, and the diastolic phase from each of the individual cardiac cycles in each of the arterial pressure waveforms of the second clinical dataset.

A further embodiment of the foregoing method, wherein the plurality of waveform signal measures of the second clinical dataset correspond to hemodynamic effects from each of the systolic rise phase, the systolic decay phase, and the diastolic phase from each of the individual cardiac cycles in each of the arterial pressure waveforms of the second clinical dataset, and wherein the hemodynamic effects comprise contractility, aortic compliance, stroke volume, vascular tone, afterload, and full cardiac cycle.

A further embodiment of the foregoing method, wherein the plurality of waveform signal measures of the second clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the second clinical dataset.

A further embodiment of the foregoing method, wherein the plurality of waveform signal measures of the second clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the second clinical dataset.

A further embodiment of the foregoing method, wherein computing the combinatorial measures between the plurality of waveform signal measures of the second clinical dataset: performing step one by arbitrarily selecting subset of signal measures from the plurality of waveform signal measures of the second clinical dataset; performing step two by calculating different orders of power for each of the subset of signal measures from the plurality of waveform signal measures of the second clinical dataset to generate powers of the subset of signal measures from the plurality of waveform signal measures of the second clinical dataset; performing step three by multiplying the powers of the subset of signal measures from the plurality of waveform signal measures of the second clinical dataset together to generate a product of the powers of the subset of signal measures from the plurality of waveform signal measures of the second clinical dataset; performing step four by performing receiver operating characteristic (ROC) analysis of the product of the powers of the subset of signal measures from the plurality of waveform signal measures of the second clinical dataset to arrive at a combinatorial measure for the subset of signal measures product of the powers of the subset of signal measures from the plurality of waveform signal measures of the second clinical dataset; and repeating steps one, two, three, and four until all of the combinatorial measures have been computed between all of the plurality of waveform signal measures of the second clinical dataset.

A further embodiment of the foregoing method, wherein performing waveform analysis of the labeled arterial pressure waveforms of the third clinical dataset to calculate the plurality of waveform signal measures of the third clinical dataset comprises: identifying individual cardiac cycles in each of the arterial pressure waveforms of the third clinical dataset; identifying a dicrotic notch in each of the individual cardiac cycles in each of the arterial pressure waveforms of the third clinical dataset; identifying a systolic rise phase, a systolic decay phase, and a diastolic phase in each of the individual cardiac cycles in each of the arterial pressure waveforms of the third clinical dataset; and extracting the plurality of waveform signal measures of the third clinical dataset from each of the systolic rise phase, the systolic decay phase, and the diastolic phase from each of the individual cardiac cycles in each of the arterial pressure waveforms of the third clinical dataset.

A further embodiment of the foregoing method, wherein the plurality of waveform signal measures of the third clinical dataset correspond to hemodynamic effects from each of the systolic rise phase, the systolic decay phase, and the diastolic phase from each of the individual cardiac cycles in each of the arterial pressure waveforms of the third clinical dataset, and wherein the hemodynamic effects comprise contractility, aortic compliance, stroke volume, vascular tone, afterload, and full cardiac cycle.

A further embodiment of the foregoing method, wherein the plurality of waveform signal measures of the third clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the third clinical dataset.

A further embodiment of the foregoing method, wherein the plurality of waveform signal measures of the third clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the third clinical dataset.

A further embodiment of the foregoing method, wherein computing the combinatorial measures between the plurality of waveform signal measures of the third clinical dataset: performing step one by arbitrarily selecting a subset of signal measures from the plurality of waveform signal measures of the third clinical dataset; performing step two by calculating different orders of power for each of the signal measures from the subset of signal measures from the plurality of waveform signal measures of the third clinical dataset to generate powers of the subset of signal measures from the plurality of waveform signal measures of the third clinical dataset; performing step three by multiplying the powers of the subset of signal measures from the plurality of waveform signal measures of the third clinical dataset together to generate a product of the powers of the subset of signal measures from the plurality of waveform signal measures of the third clinical dataset; performing step four by performing receiver operating characteristic (ROC) analysis of the product of the powers of the subset of signal measures from the plurality of waveform signal measures of the third clinical dataset to arrive at a combinatorial measure for the product of the powers of the subset of signal measures from the plurality of waveform signal measures of the third clinical dataset; and repeating steps one, two, three, and four until all of the combinatorial measures have been computed between all of the plurality of waveform signal measures of the third clinical dataset.

In another example, a method for training a hemodynamic monitor to determine an ejection fraction of a patient is disclosed. The method of training the hemodynamic monitor includes collecting a first clinical dataset containing arterial pressure waveforms from a first group of individuals with a normal ejection fraction measurement above fifty percent. A second clinical dataset is collected containing arterial pressure waveforms from a second group of individuals with a low ejection fraction measurement less than or equal to forty percent. The method further includes collecting a third clinical dataset containing arterial pressure waveforms from a third group of individuals with a borderline ejection fraction measurement within forty-one percent and forty-nine percent. Waveform analysis of the arterial pressure waveforms of the first clinical dataset, the second clinical dataset, and the third clinical dataset is performed to calculate a plurality of waveform signal measures. Input features are determined by computing combinatorial measures between the plurality of waveform signal measures and selecting top signal measures from the plurality of waveform signal measures with most predictive combinatorial measures and labeling the top signal measures as the input features. The input features are saved to a memory of the hemodynamic monitor.

The method of the preceding paragraph can optionally include, additionally and/or alternatively, any one or more of the following features, configurations, and/or additional components listed below.

A further embodiment of the foregoing method, further comprising: connecting a hemodynamic sensor to the hemodynamic monitor and to the patient to input a sensed arterial pressure waveform of the patient into the hemodynamic monitor; extracting by a processor of the hemodynamic monitor values for the input features of the sensed arterial pressure waveform of the patient; determining, by the processor of the hemodynamic monitor based on the values of the input features of the sensed arterial pressure waveform, the ejection fraction of the patient; and outputting the ejection fraction of the patient to a display and/or mobile device.

A further embodiment of the foregoing method, further comprising: alerting the patient and/or medical personnel that the ejection fraction is low when the ejection fraction is less than or equal to forty percent.

A further embodiment of the foregoing method, further comprising: alerting the patient and/or the medical personnel that the ejection fraction is borderline when the ejection fraction is within forty-one percent and forty-nine percent.

A further embodiment of the foregoing method, further comprising: alerting the patient and/or the medical personnel that the ejection fraction is normal when the ejection fraction is above fifty percent.

In another example, a hemodynamic monitor is disclosed for detecting heart failure. The hemodynamic monitor includes a non-invasive blood pressure sensor comprising an inflatable blood pressure bladder, a pressure controller pneumatically connected to the inflatable blood pressure bladder, and an optical transmitter and an optical receiver that are electrically connected to the pressure controller. The hemodynamic monitor also includes an integrated hardware unit with a system processor, a system memory, and a display with a user interface. The system memory includes instructions that, when executed by the system processor, are configured to: adjust, by the pressure controller, a pressure within the inflatable blood pressure bladder to maintain a constant volume of an artery of a patient for a period of time based on a feedback signal generated by the optical transmitter and the optical receiver; generate an arterial pressure waveform data of the patient based on the adjusted pressure within the inflatable blood pressure bladder over the period of time; extract a plurality of signal measures from the arterial pressure waveform data of the patient; extract input features from the plurality of signal measures that are indicative of an ejection fraction score of the patient; determine the ejection fraction score of the patient based on the extracted input features; generate a first sensory alarm signal configured to generate a first sensory alert that indicates that the patient has low ejection fraction when the ejection fraction score exceeds a threshold score, or generate a second sensory alarm signal configured to generate a second sensory alert that indicates that the patient does not have low ejection fraction when the ejection fraction score is below the threshold score; transmit the first sensory alarm signal or the second sensory alarm signal to the user interface; and output the first sensory alert or the second sensory alert through the user interface.

The hemodynamic monitor of the preceding paragraph can optionally include, additionally and/or alternatively, any one or more of the following features, configurations, and/or additional components listed below.

A further embodiment of the foregoing hemodynamic monitor, wherein the input features are determined by machine training, wherein the machine training comprises: collecting a first clinical dataset containing arterial pressure waveforms from a first group of individuals with a normal ejection fraction measurement above fifty percent; collecting a second clinical dataset containing arterial pressure waveforms from a second group of individuals with a low ejection fraction measurement less than or equal to forty percent; collecting a third clinical dataset containing arterial pressure waveforms from a third group of individuals with a borderline ejection fraction measurement within forty-one percent and forty-nine percent; performing waveform analysis of the arterial pressure waveforms of the first clinical dataset, the second clinical dataset, and the third clinical dataset to calculate a plurality of waveform signal measures; and determining the input features by computing combinatorial measures between the plurality of waveform signal measures and selecting top signal measures from the plurality of waveform signal measures with most predictive combinatorial measures and labeling the top signal measures as the input features.

A further embodiment of the foregoing hemodynamic monitor, wherein performing waveform analysis of the arterial pressure waveforms of the first clinical dataset, the second clinical dataset, and the third clinical dataset to calculate the plurality of waveform signal measures comprises: identifying individual cardiac cycles in each of the arterial pressure waveforms of the first clinical dataset, the second clinical dataset, and the third clinical dataset; identifying a dicrotic notch in each of the individual cardiac cycles; identifying a systolic rise phase, a systolic decay phase, and a diastolic phase in each of the individual cardiac cycles; and extracting the plurality of waveform signal measures from each of the systolic rise phase, the systolic decay phase, and the diastolic phase from each of the individual cardiac cycles.

A further embodiment of the foregoing hemodynamic monitor, wherein the plurality of waveform signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

A further embodiment of the foregoing hemodynamic monitor, wherein: the plurality of waveform signal measures comprises a mean, a maximum, a minimum, a duration, an area, a standard deviation, derivatives, and/or morphological measures from each of the systolic rise phase, the systolic decay phase, and the diastolic phase from each of the individual cardiac cycles; and/or the plurality of waveform signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

A further embodiment of the foregoing hemodynamic monitor, wherein computing the combinatorial measures between the plurality of waveform signal measures of the first clinical dataset, the second clinical dataset, and the third clinical dataset comprises: performing step one by arbitrarily selecting subset of signal measures from the plurality of waveform signal measures; performing step two by calculating different orders of power for each of the subset of signal measures to generate powers of the subset of signal measures; performing step three by multiplying the powers of the subset of signal measures together to generate the product of the powers of the subset of signal measures; performing step four by performing receiver operating characteristic (ROC) analysis of the product to arrive at a combinatorial measure for the subset of signal measures; and repeating steps one, two, three, and four until all of the combinatorial measures have been computed between all of the plurality of waveform signal measures.

A further embodiment of the foregoing hemodynamic monitor, wherein the input features comprise a first subset and a second subset, and wherein the instructions, when executed by the system processor, are further configured to: extract the first subset and the second subset of the input features concurrently from the plurality of signal measures; concurrently determine a normal ejection fraction score of the patient from the first subset of the input features and a low ejection fraction score of the patient from the second subset of the input features; output the normal ejection fraction score of the patient and the low ejection fraction score of the patient to the display of the user interface.

A further embodiment of the foregoing hemodynamic monitor, wherein the input features comprise a third subset, and wherein the instructions, when executed by the system processor, are further configured to: extract the first subset, the second subset, and the third subset of the input features concurrently from the plurality of signal measures; concurrently determine the normal ejection fraction score of the patient from the first subset of the input features, the low ejection fraction score of the patient from the second subset of the input features, and a borderline ejection fraction score of the patient from the third subset of the input features; output the normal ejection fraction score of the patient, the low ejection fraction score of the patient, and the borderline ejection fraction score of the patient to the display of the user interface.

In another example, a hemodynamic monitor is disclosed for detecting heart failure. The hemodynamic monitor includes an arterial blood pressure sensor with a housing, a fluid input port connected via tubing to a fluid source, a catheter-side fluid port connected to a catheter inserted within an arterial system of a patient, a pressure transducer in communication with the fluid source through the fluid port, and an I/O cable in electrical communication with the pressure transducer. The hemodynamic monitor also includes an integrated hardware unit with a system processor, a system memory, a display comprising a user interface, and an analog-to-digital (ADC) converter. The system memory includes instructions that, when executed by the system processor, are configured to: receive an electrical signal from the pressure transducer over a period of time, the electrical signal based on a pressure of the arterial system of the patient transmitted through the fluid source; convert the electrical signal to a digital signal; generate an arterial pressure waveform data of the patient based on the digital signal; extract a plurality of signal measures from the arterial pressure waveform data; extract input features from the plurality of signal measures that are indicative of an ejection fraction score of the patient; determine the ejection fraction score of the patient based on the extracted input features; generate a first sensory alarm signal configured to generate a first sensory alert that indicates that the patient has low ejection fraction when the ejection fraction score exceeds a threshold score, or generate a second sensory alarm signal configured to generate a second sensory alert that indicates that the patient does not have low ejection fraction when the ejection fraction score is below the threshold score; transmit the first sensory alarm signal or the second sensory alarm signal to the user interface; and output the first sensory alert or the second sensory alert through the user interface.

The hemodynamic monitor of the preceding paragraph can optionally include, additionally and/or alternatively, any one or more of the following features, configurations, and/or additional components listed below.

A further embodiment of the foregoing hemodynamic monitor, wherein the input features are determined by machine training, wherein the machine training comprises: collecting a first clinical dataset containing arterial pressure waveforms from a first group of individuals with a normal ejection fraction measurement above fifty percent; collecting a second clinical dataset containing arterial pressure waveforms from a second group of individuals with a low ejection fraction measurement less than or equal to forty percent; collecting a third clinical dataset containing arterial pressure waveforms from a third group of individuals with a borderline ejection fraction measurement within forty-one percent and forty-nine percent; performing waveform analysis of the arterial pressure waveforms of the first clinical dataset, the second clinical dataset, and the third clinical dataset to calculate a plurality of waveform signal measures; and determining the input features by computing combinatorial measures between the plurality of waveform signal measures and selecting top signal measures from the plurality of waveform signal measures with most predictive combinatorial measures and labeling the top signal measures as the input features.

A further embodiment of the foregoing hemodynamic monitor, wherein performing waveform analysis of the arterial pressure waveforms of the first clinical dataset, the second clinical dataset, and the third clinical dataset to calculate the plurality of waveform signal measures comprises: identifying individual cardiac cycles in each of the arterial pressure waveforms of the first clinical dataset, the second clinical dataset, and the third clinical dataset; identifying a dicrotic notch in each of the individual cardiac cycles; identifying a systolic rise phase, a systolic decay phase, and a diastolic phase in each of the individual cardiac cycles; and extracting the plurality of waveform signal measures from each of the systolic rise phase, the systolic decay phase, and the diastolic phase from each of the individual cardiac cycles.

A further embodiment of the foregoing hemodynamic monitor, wherein the plurality of waveform signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

A further embodiment of the foregoing hemodynamic monitor, wherein: the plurality of waveform signal measures comprises a mean, a maximum, a minimum, a duration, an area, a standard deviation, derivatives, and/or morphological measures from each of the systolic rise phase, the systolic decay phase, and the diastolic phase from each of the individual cardiac cycles; and/or the plurality of waveform signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

A further embodiment of the foregoing hemodynamic monitor, wherein computing the combinatorial measures between the plurality of waveform signal measures of the first clinical dataset, the second clinical dataset, and the third clinical dataset comprises: performing step one by arbitrarily selecting subset of signal measures from the plurality of waveform signal measures; performing step two by calculating different orders of power for each of the subset of signal measures to generate powers of the subset of signal measures; performing step three by multiplying the powers of the subset of signal measures together to generate the product of the powers of the subset of signal measures; performing step four by performing receiver operating characteristic (ROC) analysis of the product to arrive at a combinatorial measure for the subset of signal measures; and repeating steps one, two, three, and four until all of the combinatorial measures have been computed between all of the plurality of waveform signal measures.

A further embodiment of the foregoing hemodynamic monitor, wherein the input features comprise a first subset and a second subset, and wherein the instructions, when executed by the system processor, are further configured to: extract the first subset and the second subset of the input features concurrently from the plurality of signal measures; concurrently determine a normal ejection fraction score of the patient from the first subset of the input features and a low ejection fraction score of the patient from the second subset of the input features; determine the ejection fraction measurement of the patient based on the normal ejection fraction score of the patient and the low ejection fraction score of the patient; and output the ejection fraction measurement to the display of the user interface.

A further embodiment of the foregoing hemodynamic monitor, wherein the input features comprise a third subset, and wherein the instructions, when executed by the system processor, are further configured to: extract the first subset, the second subset, and the third subset of the input features concurrently from the plurality of signal measures; concurrently determine the normal ejection fraction score of the patient from the first subset of the input features, the low ejection fraction score of the patient from the second subset of the input features, and a borderline ejection fraction score of the patient from the third subset of the input features; output the normal ejection fraction score of the patient, the low ejection fraction score of the patient, and the borderline ejection fraction score of the patient to the display of the user interface.

In another example, a method is disclosed for triaging a patient for risk of heart failure. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. The method further includes extracting, by the hemodynamic monitor, input features from the plurality of signal measures that are indicative of an ejection fraction of the patient. Extracting the input features includes extracting a first subset of the input features and extracting a second subset of the input features concurrently with the first subset of the input features. The method further includes concurrently determining, by the hemodynamic monitor, a normal ejection fraction score of the patient from the first subset of the input features and a low ejection fraction score of the patient from the second subset of the input features. The hemodynamic monitor outputs the normal ejection fraction score and the low ejection fraction score of the patient to a display and/or mobile device.

The method of the preceding paragraph can optionally include, additionally and/or alternatively, any one or more of the following features, configurations, and/or additional components listed below.

A further embodiment of the foregoing method, wherein extracting the input features further comprises: extracting a third subset of the input features concurrently with the first subset and the second subset of the input features; and wherein the hemodynamic monitor concurrently determines the normal ejection fraction score of the patient from the first subset of the input features, the low ejection fraction score of the patient from the second subset of the input features, and a borderline ejection fraction score of the patient from the third subset of the input features; and wherein the hemodynamic monitor outputs the normal ejection fraction score of the patient, the low ejection fraction score of the patient, and the borderline ejection fraction score of the patient to the display and/or the mobile device.

A further embodiment of the foregoing method, further comprising: training the hemodynamic monitor for determining the ejection fraction of the patient, wherein training the hemodynamic monitor comprises: collecting a first clinical dataset containing arterial pressure waveforms from a first group of individuals with a normal ejection fraction measurement above fifty percent; labeling each of the arterial pressure waveforms of the first clinical dataset with a first label; performing waveform analysis of the labeled arterial pressure waveforms of the first clinical dataset to calculate a plurality of waveform signal measures of the first clinical dataset; determining a first subset of the input features by computing combinatorial measures between the plurality of waveform signal measures of the first clinical dataset and selecting top signal measures from the plurality of waveform signal measures of the first clinical dataset with most predictive combinatorial measures and labeling the top signal measures of the first clinical data set as the first subset of the input features; collecting a second clinical dataset containing arterial pressure waveforms from a second group of individuals with a low ejection fraction measurement less than or equal to forty percent; labeling each of the arterial pressure waveforms of the second clinical dataset with a second label; performing waveform analysis of the labeled arterial pressure waveforms of the second clinical dataset to calculate a plurality of waveform signal measures of the second clinical dataset; determining a second subset of the input features by computing combinatorial measures between the plurality of waveform signal measures of the second clinical dataset and selecting top signal measures from the plurality of waveform signal measures of the second clinical dataset with most predictive combinatorial measures and labeling the top signal measures of the second clinical data set as the second subset of the input features; collecting a third clinical dataset containing arterial pressure waveforms from a third group of individuals with a borderline ejection fraction measurement within forty-one percent and forty-nine percent; labeling each of the arterial pressure waveforms of the third clinical dataset with a third label; performing waveform analysis of the labeled arterial pressure waveforms of the third clinical dataset to calculate a plurality of waveform signal measures of the third clinical dataset; and determining a third subset of the input features by computing combinatorial measures between the plurality of waveform signal measures of the third clinical dataset and selecting top signal measures from the plurality of waveform signal measures of the third clinical dataset with most predictive combinatorial measures and labeling the top signal measures of the third clinical data set as the third subset of the input features.

A further embodiment of the foregoing method, wherein performing waveform analysis of the labeled arterial pressure waveforms of the first clinical dataset to calculate the plurality of waveform signal measures of the first clinical dataset comprises: identifying individual cardiac cycles in each of the arterial pressure waveforms of the first clinical dataset; identifying a dicrotic notch in each of the individual cardiac cycles in each of the arterial pressure waveforms of the first clinical dataset; identifying a systolic rise phase, a systolic decay phase, and a diastolic phase in each of the individual cardiac cycles in each of the arterial pressure waveforms of the first clinical dataset; and extracting the plurality of waveform signal measures of the first clinical dataset from each of the systolic rise phase, the systolic decay phase, and the diastolic phase from each of the individual cardiac cycles in each of the arterial pressure waveforms of the first clinical dataset.

A further embodiment of the foregoing method, wherein performing waveform analysis of the labeled arterial pressure waveforms of the second clinical dataset to calculate the plurality of waveform signal measures of the second clinical dataset comprises: identifying individual cardiac cycles in each of the arterial pressure waveforms of the second clinical dataset; identifying a dicrotic notch in each of the individual cardiac cycles in each of the arterial pressure waveforms of the second clinical dataset; identifying a systolic rise phase, a systolic decay phase, and a diastolic phase in each of the individual cardiac cycles in each of the arterial pressure waveforms of the second clinical dataset; and extracting the plurality of waveform signal measures of the second clinical dataset from each of the systolic rise phase, the systolic decay phase, and the diastolic phase from each of the individual cardiac cycles in each of the arterial pressure waveforms of the second clinical dataset.

A further embodiment of the foregoing method, wherein performing waveform analysis of the labeled arterial pressure waveforms of the third clinical dataset to calculate the plurality of waveform signal measures of the third clinical dataset comprises: identifying individual cardiac cycles in each of the arterial pressure waveforms of the third clinical dataset; identifying a dicrotic notch in each of the individual cardiac cycles in each of the arterial pressure waveforms of the third clinical dataset; identifying a systolic rise phase, a systolic decay phase, and a diastolic phase in each of the individual cardiac cycles in each of the arterial pressure waveforms of the third clinical dataset; and extracting the plurality of waveform signal measures of the third clinical dataset from each of the systolic rise phase, the systolic decay phase, and the diastolic phase from each of the individual cardiac cycles in each of the arterial pressure waveforms of the third clinical dataset.

A further embodiment of the foregoing method, wherein: the plurality of waveform signal measures of the first clinical dataset correspond to hemodynamic effects from each of the systolic rise phase, the systolic decay phase, and the diastolic phase from each of the individual cardiac cycles in each of the arterial pressure waveforms of the first clinical dataset, and wherein the hemodynamic effects comprise contractility, aortic compliance, stroke volume, vascular tone, afterload, and full cardiac cycle; the plurality of waveform signal measures of the second clinical dataset correspond to hemodynamic effects from each of the systolic rise phase, the systolic decay phase, and the diastolic phase from each of the individual cardiac cycles in each of the arterial pressure waveforms of the second clinical dataset, and wherein the hemodynamic effects comprise contractility, aortic compliance, stroke volume, vascular tone, afterload, and full cardiac cycle; and the plurality of waveform signal measures of the third clinical dataset correspond to hemodynamic effects from each of the systolic rise phase, the systolic decay phase, and the diastolic phase from each of the individual cardiac cycles in each of the arterial pressure waveforms of the third clinical dataset, and wherein the hemodynamic effects comprise contractility, aortic compliance, stroke volume, vascular tone, afterload, and full cardiac cycle.

A further embodiment of the foregoing method, wherein: the plurality of waveform signal measures of the first clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the first clinical dataset; the plurality of waveform signal measures of the second clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the second clinical dataset; and the plurality of waveform signal measures of the third clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the third clinical dataset.

A further embodiment of the foregoing method, wherein: the plurality of waveform signal measures of the first clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the first clinical dataset; the plurality of waveform signal measures of the second clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the second clinical dataset; and wherein the plurality of waveform signal measures of the third clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the third clinical dataset.

A further embodiment of the foregoing method, wherein computing the combinatorial measures between the plurality of waveform signal measures of the first clinical dataset comprises: performing step one by arbitrarily selecting subset of signal measures from the plurality of waveform signal measures of the first clinical dataset; performing step two by calculating different orders of power for each of the subset of signal measures from the plurality of waveform signal measures of the first clinical dataset to generate powers of the subset of signal measures from the plurality of waveform signal measures of the first clinical dataset; performing step three by multiplying the powers of the subset of signal measures from the plurality of waveform signal measures of the first clinical dataset together to generate a product of the powers of the subset of signal measures from the plurality of waveform signal measures of the first clinical dataset; performing step four by performing receiver operating characteristic (ROC) analysis of the product of the powers of the subset of signal measures from the plurality of waveform signal measures of the first clinical dataset to arrive at a combinatorial measure for the subset of signal measures product of the powers of the subset of signal measures from the plurality of waveform signal measures of the first clinical dataset; and repeating steps one, two, three, and four until all of the combinatorial measures have been computed between all of the plurality of waveform signal measures of the first clinical dataset.

A further embodiment of the foregoing method, wherein computing the combinatorial measures between the plurality of waveform signal measures of the second clinical dataset comprises: performing step one by arbitrarily selecting subset of signal measures from the plurality of waveform signal measures of the second clinical dataset; performing step two by calculating different orders of power for each of the subset of signal measures from the plurality of waveform signal measures of the second clinical dataset to generate powers of the subset of signal measures from the plurality of waveform signal measures of the second clinical dataset; performing step three by multiplying the powers of the subset of signal measures from the plurality of waveform signal measures of the second clinical dataset together to generate a product of the powers of the subset of signal measures from the plurality of waveform signal measures of the second clinical dataset; performing step four by performing receiver operating characteristic (ROC) analysis of the product of the powers of the subset of signal measures from the plurality of waveform signal measures of the second clinical dataset to arrive at a combinatorial measure for the subset of signal measures product of the powers of the subset of signal measures from the plurality of waveform signal measures of the second clinical dataset; and repeating steps one, two, three, and four until all of the combinatorial measures have been computed between all of the plurality of waveform signal measures of the second clinical dataset; wherein computing the combinatorial measures between the plurality of waveform signal measures of the third clinical dataset comprises: performing step one by arbitrarily selecting a subset of signal measures from the plurality of waveform signal measures of the third clinical dataset; performing step two by calculating different orders of power for each of the signal measures from the subset of signal measures from the plurality of waveform signal measures of the third clinical dataset to generate powers of the subset of signal measures from the plurality of waveform signal measures of the third clinical dataset; performing step three by multiplying the powers of the subset of signal measures from the plurality of waveform signal measures of the third clinical dataset together to generate a product of the powers of the subset of signal measures from the plurality of waveform signal measures of the third clinical dataset; performing step four by performing receiver operating characteristic (ROC) analysis of the product of the powers of the subset of signal measures from the plurality of waveform signal measures of the third clinical dataset to arrive at a combinatorial measure for the product of the powers of the subset of signal measures from the plurality of waveform signal measures of the third clinical dataset; and repeating steps one, two, three, and four until all of the combinatorial measures have been computed between all of the plurality of waveform signal measures of the third clinical dataset.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A hemodynamic monitor for detecting heart failure, the hemodynamic monitor comprising:
a non-invasive blood pressure sensor comprising an inflatable blood pressure bladder, a pressure controller pneumatically connected to the inflatable blood pressure bladder, and an optical transmitter and an optical receiver that are electrically connected to the pressure controller;
an integrated hardware unit comprising:
a system processor;
a system memory; and
a display comprising a user interface; and
wherein the system memory comprises instructions that, when executed by the system processor, are configured to:
adjust, by the pressure controller, a pressure within the inflatable blood pressure bladder to maintain a constant volume of an artery of a patient for a period of time based on a feedback signal generated by the optical transmitter and the optical receiver;
generate an arterial pressure waveform data of the patient based on the adjusted pressure within the inflatable blood pressure bladder over the period of time;
extract a plurality of signal measures from the arterial pressure waveform data of the patient;
extract input features from the plurality of signal measures that are indicative of an ejection fraction score of the patient;
determine the ejection fraction score of the patient based on the extracted input features;
generate a first sensory alarm signal configured to generate a first sensory alert that indicates that the patient has low ejection fraction when the ejection fraction score exceeds a threshold score, or generate a second sensory alarm signal configured to generate a second sensory alert that indicates that the patient does not have low ejection fraction when the ejection fraction score is below the threshold score;
transmit the first sensory alarm signal or the second sensory alarm signal to the user interface; and
output the first sensory alert or the second sensory alert through the user interface.

2. The hemodynamic monitor of claim 1, wherein the input features are determined by machine training, wherein the machine training comprises:
collecting a first clinical dataset containing arterial pressure waveforms from a first group of individuals with a normal ejection fraction measurement above fifty percent;
collecting a second clinical dataset containing arterial pressure waveforms from a second group of individuals with a low ejection fraction measurement less than or equal to forty percent;
collecting a third clinical dataset containing arterial pressure waveforms from a third group of individuals with a borderline ejection fraction measurement within forty-one percent and forty-nine percent;
performing waveform analysis of the arterial pressure waveforms of the first clinical dataset, the second clinical dataset, and the third clinical dataset to calculate a plurality of waveform signal measures; and
determining the input features by computing combinatorial measures between the plurality of waveform signal measures and selecting top signal measures from the plurality of waveform signal measures with most predictive combinatorial measures and labeling the top signal measures as the input features.

3. The hemodynamic monitor of claim 2, wherein performing waveform analysis of the arterial pressure waveforms of the first clinical dataset, the second clinical dataset, and the third clinical dataset to calculate the plurality of waveform signal measures comprises:
identifying individual cardiac cycles in each of the arterial pressure waveforms of the first clinical dataset, the second clinical dataset, and the third clinical dataset;
identifying a dicrotic notch in each of the individual cardiac cycles;
identifying a systolic rise phase, a systolic decay phase, and a diastolic phase in each of the individual cardiac cycles; and
extracting the plurality of waveform signal measures from each of the systolic rise phase, the systolic decay phase, and the diastolic phase from each of the individual cardiac cycles.

4. The hemodynamic monitor of claim 3, wherein the plurality of waveform signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

5. The hemodynamic monitor of claim 4, wherein:
the plurality of waveform signal measures comprises a mean, a maximum, a minimum, a duration, an area, a standard deviation, derivatives, and/or morphological measures from each of the systolic rise phase, the systolic decay phase, and the diastolic phase from each of the individual cardiac cycles; and/or
the plurality of waveform signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

6. The hemodynamic monitor of claim 5, wherein computing the combinatorial measures between the plurality of waveform signal measures of the first clinical dataset, the second clinical dataset, and the third clinical dataset comprises:
performing step one by arbitrarily selecting subset of signal measures from the plurality of waveform signal measures;
performing step two by calculating different orders of power for each of the subset of signal measures to generate powers of the subset of signal measures;
performing step three by multiplying the powers of the subset of signal measures together to generate the product of the powers of the subset of signal measures;
performing step four by performing receiver operating characteristic (ROC) analysis of the product to arrive at a combinatorial measure for the subset of signal measures; and
repeating steps one, two, three, and four until all of the combinatorial measures have been computed between all of the plurality of waveform signal measures.

7. The hemodynamic monitor of claim 6, wherein the input features comprise a first subset and a second subset, and wherein the instructions, when executed by the system processor, are further configured to:
extract the first subset and the second subset of the input features concurrently from the plurality of signal measures;
concurrently determine a normal ejection fraction score of the patient from the first subset of the input features and a low ejection fraction score of the patient from the second subset of the input features;
output the normal ejection fraction score of the patient and the low ejection fraction score of the patient to the display of the user interface.

8. The hemodynamic monitor of claim 7, wherein the input features comprise a third subset, and wherein the instructions, when executed by the system processor, are further configured to:
extract the first subset, the second subset, and the third subset of the input features concurrently from the plurality of signal measures;
concurrently determine the normal ejection fraction score of the patient from the first subset of the input features, the low ejection fraction score of the patient from the second subset of the input features, and a borderline ejection fraction score of the patient from the third subset of the input features; and
output the normal ejection fraction score of the patient, the low ejection fraction score of the patient, and the borderline ejection fraction score of the patient to the display of the user interface.

9. A method for triaging a patient for risk of heart failure, the method comprising:
receiving, by a hemodynamic monitor, sensed hemodynamic data representative of an arterial pressure waveform of the patient;
performing, by the hemodynamic monitor, waveform analysis of the sensed hemodynamic data to calculate a plurality of signal measures of the sensed hemodynamic data;
extracting, by the hemodynamic monitor, input features from the plurality of signal measures that are indicative of an ejection fraction of the patient, wherein extracting the input features comprises:
extracting a first subset of the input features; and
extracting a second subset of the input features concurrently with the first subset of the input features;
concurrently determining, by the hemodynamic monitor, a normal ejection fraction score of the patient from the first subset of the input features, and a low ejection fraction score of the patient from the second subset of the input features; and
outputting the normal ejection fraction score and the low ejection fraction score of the patient to a display and/or mobile device.

10. The method of claim 9, wherein extracting the input features further comprises:
extracting a third subset of the input features concurrently with the first subset and the second subset of the input features; and
wherein the hemodynamic monitor concurrently determines the normal ejection fraction score of the patient from the first subset of the input features, the low ejection fraction score of the patient from the second subset of the input features, and a borderline ejection fraction score of the patient from the third subset of the input features; and
wherein the hemodynamic monitor outputs the normal ejection fraction score of the patient, the low ejection fraction score of the patient, and the borderline ejection fraction score of the patient to the display and/or the mobile device.

11. The method of claim 10, further comprising:
training the hemodynamic monitor for determining the ejection fraction of the patient, wherein training the hemodynamic monitor comprises:
collecting a first clinical dataset containing arterial pressure waveforms from a first group of individuals with a normal ejection fraction measurement above fifty percent;
labeling each of the arterial pressure waveforms of the first clinical dataset with a first label;
performing waveform analysis of the labeled arterial pressure waveforms of the first clinical dataset to calculate a plurality of waveform signal measures of the first clinical dataset;
determining a first subset of the input features by computing combinatorial measures between the plurality of waveform signal measures of the first clinical dataset and selecting top signal measures from the plurality of waveform signal measures of the first clinical dataset with most predictive combinatorial measures and labeling the top signal measures of the first clinical data set as the first subset of the input features;
collecting a second clinical dataset containing arterial pressure waveforms from a second group of individuals with a low ejection fraction measurement less than or equal to forty percent;
labeling each of the arterial pressure waveforms of the second clinical dataset with a second label;
performing waveform analysis of the labeled arterial pressure waveforms of the second clinical dataset to calculate a plurality of waveform signal measures of the second clinical dataset;
determining a second subset of the input features by computing combinatorial measures between the plurality of waveform signal measures of the second clinical dataset and selecting top signal measures from the plurality of waveform signal measures of the second clinical dataset with most predictive combinatorial measures and labeling the top signal measures of the second clinical data set as the second subset of the input features;
collecting a third clinical dataset containing arterial pressure waveforms from a third group of individuals with a borderline ejection fraction measurement within forty-one percent and forty-nine percent;
labeling each of the arterial pressure waveforms of the third clinical dataset with a third label;
performing waveform analysis of the labeled arterial pressure waveforms of the third clinical dataset to calculate a plurality of waveform signal measures of the third clinical dataset; and
determining a third subset of the input features by computing combinatorial measures between the plurality of waveform signal measures of the third clinical dataset and selecting top signal measures from the plurality of waveform signal measures of the third clinical dataset with most predictive combinatorial measures and labeling the top signal measures of the third clinical data set as the third subset of the input features.

12. The method of claim 11, wherein performing waveform analysis of the labeled arterial pressure waveforms of the first clinical dataset to calculate the plurality of waveform signal measures of the first clinical dataset comprises:
identifying individual cardiac cycles in each of the arterial pressure waveforms of the first clinical dataset;
identifying a dicrotic notch in each of the individual cardiac cycles in each of the arterial pressure waveforms of the first clinical dataset;
identifying a systolic rise phase, a systolic decay phase, and a diastolic phase in each of the individual cardiac cycles in each of the arterial pressure waveforms of the first clinical dataset; and
extracting the plurality of waveform signal measures of the first clinical dataset from each of the systolic rise phase, the systolic decay phase, and the diastolic phase from each of the individual cardiac cycles in each of the arterial pressure waveforms of the first clinical dataset.

13. The method of claim 12, wherein performing waveform analysis of the labeled arterial pressure waveforms of the second clinical dataset to calculate the plurality of waveform signal measures of the second clinical dataset comprises:
identifying individual cardiac cycles in each of the arterial pressure waveforms of the second clinical dataset;
identifying a dicrotic notch in each of the individual cardiac cycles in each of the arterial pressure waveforms of the second clinical dataset;
identifying a systolic rise phase, a systolic decay phase, and a diastolic phase in each of the individual cardiac cycles in each of the arterial pressure waveforms of the second clinical dataset; and
extracting the plurality of waveform signal measures of the second clinical dataset from each of the systolic rise phase, the systolic decay phase, and the diastolic phase from each of the individual cardiac cycles in each of the arterial pressure waveforms of the second clinical dataset.

14. The method of claim 13, wherein performing waveform analysis of the labeled arterial pressure waveforms of the third clinical dataset to calculate the plurality of waveform signal measures of the third clinical dataset comprises:
identifying individual cardiac cycles in each of the arterial pressure waveforms of the third clinical dataset;
identifying a dicrotic notch in each of the individual cardiac cycles in each of the arterial pressure waveforms of the third clinical dataset;
identifying a systolic rise phase, a systolic decay phase, and a diastolic phase in each of the individual cardiac cycles in each of the arterial pressure waveforms of the third clinical dataset; and
extracting the plurality of waveform signal measures of the third clinical dataset from each of the systolic rise phase, the systolic decay phase, and the diastolic phase from each of the individual cardiac cycles in each of the arterial pressure waveforms of the third clinical dataset.

15. The method of claim 14, wherein:
the plurality of waveform signal measures of the first clinical dataset correspond to hemodynamic effects from each of the systolic rise phase, the systolic decay phase, and the diastolic phase from each of the individual cardiac cycles in each of the arterial pressure waveforms of the first clinical dataset, and wherein the hemodynamic effects comprise contractility, aortic compliance, stroke volume, vascular tone, afterload, and full cardiac cycle;
the plurality of waveform signal measures of the second clinical dataset correspond to hemodynamic effects from each of the systolic rise phase, the systolic decay phase, and the diastolic phase from each of the individual cardiac cycles in each of the arterial pressure waveforms of the second clinical dataset, and wherein the hemodynamic effects comprise contractility, aortic compliance, stroke volume, vascular tone, afterload, and full cardiac cycle; and
the plurality of waveform signal measures of the third clinical dataset correspond to hemodynamic effects from each of the systolic rise phase, the systolic decay phase, and the diastolic phase from each of the individual cardiac cycles in each of the arterial pressure waveforms of the third clinical dataset, and wherein the hemodynamic effects comprise contractility, aortic compliance, stroke volume, vascular tone, afterload, and full cardiac cycle.

16. The method of claim 15, wherein:
the plurality of waveform signal measures of the first clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the first clinical dataset;
the plurality of waveform signal measures of the second clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the second clinical dataset; and
the plurality of waveform signal measures of the third clinical dataset comprises a mean, a maximum, a minimum, a duration, an area, a standard deviation, derivatives, and/or morphological measures from each of the systolic rise phase, the systolic decay phase, and the diastolic phase from each of the individual cardiac cycles in each of the arterial pressure waveforms of the third clinical dataset.

17. The method of claim 16, wherein:
the plurality of waveform signal measures of the first clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the first clinical dataset;
the plurality of waveform signal measures of the second clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the second clinical dataset; and
wherein the plurality of waveform signal measures of the third clinical dataset comprises heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles in each of the arterial pressure waveforms of the third clinical dataset.

18. The method of claim 17, wherein computing the combinatorial measures between the plurality of waveform signal measures of the first clinical dataset comprises:
performing step one by arbitrarily selecting subset of signal measures from the plurality of waveform signal measures of the first clinical dataset;
performing step two by calculating different orders of power for each of the subset of signal measures from the plurality of waveform signal measures of the first clinical dataset to generate powers of the subset of signal measures from the plurality of waveform signal measures of the first clinical dataset;
performing step three by multiplying the powers of the subset of signal measures from the plurality of waveform signal measures of the first clinical dataset together to generate a product of the powers of the subset of signal measures from the plurality of waveform signal measures of the first clinical dataset;
performing step four by performing receiver operating characteristic (ROC) analysis of the product of the powers of the subset of signal measures from the plurality of waveform signal measures of the first clinical dataset to arrive at a combinatorial measure for the subset of signal measures product of the powers of the subset of signal measures from the plurality of waveform signal measures of the first clinical dataset; and
repeating steps one, two, three, and four until all of the combinatorial measures have been computed between all of the plurality of waveform signal measures of the first clinical dataset.

19. The method of claim 18, wherein computing the combinatorial measures between the plurality of waveform signal measures of the second clinical dataset comprises:
performing step one by arbitrarily selecting subset of signal measures from the plurality of waveform signal measures of the second clinical dataset;
performing step two by calculating different orders of power for each of the subset of signal measures from the plurality of waveform signal measures of the second clinical dataset to generate powers of the subset of signal measures from the plurality of waveform signal measures of the second clinical dataset;
performing step three by multiplying the powers of the subset of signal measures from the plurality of waveform signal measures of the second clinical dataset together to generate a product of the powers of the subset of signal measures from the plurality of waveform signal measures of the second clinical dataset;
performing step four by performing receiver operating characteristic (ROC) analysis of the product of the powers of the subset of signal measures from the plurality of waveform signal measures of the second clinical dataset to arrive at a combinatorial measure for the subset of signal measures product of the powers of the subset of signal measures from the plurality of waveform signal measures of the second clinical dataset; and
repeating steps one, two, three, and four until all of the combinatorial measures have been computed between all of the plurality of waveform signal measures of the second clinical dataset;
wherein computing the combinatorial measures between the plurality of waveform signal measures of the third clinical dataset comprises:
performing step one by arbitrarily selecting a subset of signal measures from the plurality of waveform signal measures of the third clinical dataset;
performing step two by calculating different orders of power for each of the signal measures from the subset of signal measures from the plurality of waveform signal measures of the third clinical dataset to generate powers of the subset of signal measures from the plurality of waveform signal measures of the third clinical dataset;
performing step three by multiplying the powers of the subset of signal measures from the plurality of waveform signal measures of the third clinical dataset together to generate a product of the powers of the subset of signal measures from the plurality of waveform signal measures of the third clinical dataset;
performing step four by performing receiver operating characteristic (ROC) analysis of the product of the powers of the subset of signal measures from the plurality of waveform signal measures of the third clinical dataset to arrive at a combinatorial measure for the product of the powers of the subset of signal measures from the plurality of waveform signal measures of the third clinical dataset; and
repeating steps one, two, three, and four until all of the combinatorial measures have been computed between all of the plurality of waveform signal measures of the third clinical dataset.
